(19) United States
(12) Patent Application Publication
(10) Pub. No.: US 2026/0100244 A1
(43) Pub. Date: Apr. 9, 2026

(54) PROTEIN STRUCTURE PREDICTION

(71) Applicant: Biomap (Beijing) Intelligence Technology Ltd., Beijing (CN)

(72) Inventors: Le SONG, Palo Alto, CA (US); Yining WANG, Beijing (CN); Xumeng GONG, Beijing (CN); Shaochuan LI, Beijing (CN); Bing YANG, Beijing (CN); Hui LI, Beijing (CN)

(21) Appl. No.: 19/113,308

(22) PCT Filed: Sep. 28, 2023
(86) PCT No.: PCT/US2023/034001

§ 371 Date: Mar. 19, 2025

Related U.S. Application Data
(60) Provisional application No. 63/411,526, filed on Sep. 29, 2022.
(60) Provisional application No. 63/435,529, filed on Dec. 27, 2022.

(51) Int. Cl. G16B 15/20; G16B 30/00; G16B 40/00

(52) U.S. Cl. CPC G16B 15/20; G16B 30/00; G16B 40/00

(57) ABSTRACT

Disclosed herein are methods, systems, and apparatus, including computer programs encoded on computer storage media, for antibody structure prediction. In an example method, a target antibody sequence of a target antibody that includes a sequence of amino acids is received. The target antibody sequence is processed by an antibody language model (ALM) to obtain a residue encoding and an attention weight encoding without performing multiple sequence alignment (MSA), wherein the ALM is a protein language model trained from antibody sequences, and the ALM comprises a plurality of self-attention layers. The residue encoding and the attention weight encoding are transformed into a single representation and a pair representation that are input into a structure prediction model. A predicted structure of the target antibody is determined using the structure prediction model.

### TECHNICAL FIELD

This specification relates to protein structure prediction, such as, antibody structure prediction based on machine learning technologies.

### BACKGROUND

Protein structure prediction is the inference of the three-dimensional (3D) structure of a protein from its amino acid sequence. Machine learning methods, such as deep learning methods, can be used for protein structure prediction. Deep learning methods incorporate evolutional and geometric information of protein structures and deep neural networks. In these deep learning methods, progress has been made by using the co-evolution information from Multiple Sequence Alignments (MSAs), such as AlphaFold, AlphaFold2, OpenFold, and RoseTTAFold. For example, AlphaFold2 provides an architecture to jointly model MSAs and pairwise information, and to predict protein structure based on protein sequences and MSAs. However, these methods are time-consuming and dependent on MSAs, which remains a challenge for the structure prediction of orphan proteins with less homologous information or antibody for which MSAs are not always useful on account of a fast-evolving nature.

Recently, protein structure prediction have been made on large protein language models (PLMs) which are no longer dependent on MSAs. In particular, models like DeepAb, ABlooper, and IgFold are developed for antibody structure prediction. These models can reduce computation time but incur a certain loss of prediction precision.

Techniques for efficient and accurate antibody structure prediction are desirable.

### SUMMARY

Described embodiments of the subject matter can include one or more features, alone or in combination.

For example, in one embodiment, a computer-implemented method for antibody structure prediction includes receiving, by a data processing apparatus, a target antibody sequence of a target antibody that includes a sequence of amino acids; inputting, by the data processing apparatus, the target antibody sequence into an antibody language model (ALM), wherein the ALM is a protein language model trained from antibody sequences, and the ALM comprises a plurality of self-attention layers; obtaining, by the data processing apparatus using the ALM without performing multiple sequence alignment (MSA), a residue encoding and an attention weight encoding, wherein the residue encoding comprises a respective first embedding corresponding to each of the amino acids in the target antibody sequence output by the ALM; and the attention weight encoding comprises a respective second embedding corresponding to a pair of amino acids in the target antibody sequence calculated from attention weights of the self-attention layers of the ALM; transforming, by the data processing apparatus, the residue encoding and the attention weight encoding into a single representation and a pair representation; inputting, by the data processing apparatus, the single representation and the pair representation into a structure prediction model, wherein parameters of the structure prediction model are trained based on a loss function reflecting a difference between a predicted structure and an actual structure of an antibody; determining, by the data processing apparatus, the predicted structure of the target antibody using the structure prediction model based on the single representation and the pair representation; and outputting, by the data processing apparatus, the predicted structure of the target antibody.

In some embodiments, these general and specific aspects may be implemented using a system, a method, or a computer program, or any combination of systems, methods, and computer programs. The foregoing and other described embodiments can each, optionally, include one or more of the following aspects:

In some embodiments, wherein the ALM is pre-trained using an antibody database according to a Bidirectional Encoder Representations from Transformers (BERT) architecture, and the antibody database consists the antibody sequences.

In some embodiments, wherein the ALM comprises L self-attention layers, each of the L self-attention layers comprises H attention heads, and wherein a second embedding qij corresponding to a pair of an amino acid i and an amino acid j in the target antibody sequence, and wherein obtaining, by the data processing apparatus using the ALM without performing MSA, the attention weight encoding comprises: obtaining attention weights of the H attention heads of the each of the L self-attention layers when the amino acid i is used as a query and the amino acid j is used as a key; and concatenating the attention weights to obtain the second embedding qij.

In some embodiments, wherein transforming, by the data processing apparatus, the residue encoding and the attention weight encoding into a single representation and a pair representation comprises: transforming the residue encoding into the single representation by a first linear neural network layer; and transforming the attention weight encoding into the pair representation by a second linear neural network layer; wherein parameters of the first linear neural network layer and the second linear neural network layer are updated based on a gradient of the loss function.

In some embodiments, wherein the loss function does not comprise a loss due to MSA.

In some embodiments, wherein the loss function comprises a framed aligned point error (FAPE) loss and a torsion angle loss, and a loss focusing on a complementarity determining region (CDR).

In some embodiments, wherein the loss function comprises a differential root-mean-squared-deviation (RMSD) in addition to a framed aligned point error (FAPE) loss.

In some embodiments, wherein the single representation and the pair representation do not incorporate template features before the single representation and the pair representation are input into the structure prediction model.

In some embodiments, wherein, before inputting, by the data processing apparatus, the single representation and the pair representation into the structure prediction model, the computer-implemented method further comprises: performing, by the data processing apparatus, a template search, based on the target antibody sequence without multiple sequence alignment (MSA), for one or more template candidates that have similar structures to a structure of the target antibody; and obtaining, by the data processing apparatus, template features based on the one or more template candidates; and wherein transforming, by the data processing apparatus, the residue encoding and the attention weight encoding into the single representation and the pair representation comprises: transforming, by the data processing apparatus, the residue encoding and the attention weight encoding into a preliminary single representation and an preliminary pair representation; incorporating, by the data processing apparatus, the template features into the preliminary single representation and the preliminary pair representation to obtain the single representation and the pair representation.

In some embodiments, wherein performing, by the data processing apparatus, the template search for one or more template candidates comprises: performing, by the data processing apparatus, a sequential modal search in a first structure database for first structure templates, wherein sequences of antibodies corresponding to the first structure templates are similar to the target antibody sequence; and performing, by the data processing apparatus, a structural modal search in a second structure database for second structure templates, wherein structures of the second structure templates are similar to a coarse-grained structure of the target antibody sequence, and wherein the one or more template candidates comprise one or more of the first structure templates or the second structure templates, and wherein the coarse-grained structure is a default structure or a structure predicted from another structure prediction algorithm or another structure prediction model.

It is appreciated that methods in accordance with this specification may include any combination of the aspects and features described herein. That is, methods in accordance with this specification are not limited to the combinations of aspects and features specifically described herein but also include any combination of the aspects and features provided.

The details of one or more embodiments of this specification are set forth in the accompanying drawings and the description below. Other features and advantages of this specification will be apparent from the description and drawings, and from the claims.

### BRIEF DESCRIPTION OF THE DRAWINGS

FIG. 1 is a diagram illustrating an example computer-implemented system configured for protein structure prediction, in accordance with embodiments of this specification.

FIG. 2 is a diagram illustrating an example input and output of an ALM, in accordance with embodiments of this specification.

FIG. 3 is a diagram illustrating an example residue2pair communication in an example computer-implemented system configured for protein structure prediction, in accordance with embodiments of this specification.

FIG. 4 is a table illustrating statistics of example datasets used for protein structure prediction, in accordance with embodiments of this specification.

FIG. 5 is a table illustrating accuracy performances of different example protein structure prediction models on antibody structure prediction, in accordance with embodiments of this specification.

FIG. 6 are two tables illustrating accuracy performances of different example protein structure prediction models on complementarity determining region (CDR) loop structure prediction, in accordance with embodiments of this specification.

FIG. 7 is a plot illustrating examples of protein structures predicted by an example computer-implemented system configured for protein structure prediction and other baselines, in accordance with embodiments of this specification.

FIG. 8 is a plot illustrating examples of protein structures predicted by xTrimoABFold and other baselines, in accordance with embodiments of this specification.

FIG. 9 is a graph illustrating an example experiment result with respect to antibody structure prediction performance of an example computer-implemented system configured for protein structure prediction with and without focal loss, in accordance with embodiments of this specification.

FIG. 10 is a diagram illustrating another example computer-implemented system configured for protein structure prediction, in accordance with embodiments of this specification.

FIG. 11 is another table illustrating accuracy performances of different example protein structure prediction models on antibody structure prediction, in accordance with embodiments of this specification.

FIG. 12 is a plot illustrating examples of protein structures predicted by the xTrimoABFold++ and other baselines, in accordance with embodiments of this specification.

FIG. 13 is a flowchart of an example of a process for protein structure prediction, in accordance with embodiments of this specification.

FIG. 14 is a block diagram illustrating an example of a computer-implemented system used to provide computational functionalities associated with described algorithms, methods, functions, processes, flows, and procedures, according to an embodiment of the present disclosure.

FIG. 15 depicts examples of modules of an apparatus in accordance with embodiments of this specification.

Like reference numbers and designations in the various drawings indicate like elements.

### DETAILED DESCRIPTION

This specification describes techniques for protein structure prediction, such as, antibody structure prediction, based on machine learning or artificial intelligence (AI) technologies. The described techniques can be applied, for example, in the field of antibody engineering, drug design and/or discovery, etc.

In some embodiments, techniques are described for predicting, interfering or otherwise identifying structure of proteins, especially structure of antibodies. A protein can be defined or specified by one or more amino acid chains or sequences in a 2-dimension (2D), 3-dimension (3D) or a higher-dimension. The amino acid sequences can include, for example, long polypeptides, short polypeptides, or peptides. The amino acids may be referred to as amino acid residues or simply residues when the amino acids are linked by peptide bonds in a sequence. Accordingly, a sequence or chain of amino acids is also referred to as an amino acid sequence or a residue sequence.

The structure of a protein defines a three-dimensional (3D) configuration of atoms in the amino acid sequence of the protein. In some embodiments, the structure of the protein can be defined or represented by values of structure parameters such as positions and angles of the atoms in the amino acid sequence of the protein. For example, the structure parameters of a protein can include 3D coordinates of atoms and/or relative translation and rotation between atoms in the protein.

An antibody can include, for example, a protein used by an immune system to identify and neutralize foreign objects such as pathogenic bacteria and viruses. The antibody recognizes or otherwise corresponds to an antigen. For example, an antibody can include one or more paratopes, wherein each paratope is specific for one particular epitope on an antigen, allowing these two structures to bind together with precision. In this application, the term “antigen” or “antibody” can be broad enough to encompass one or more of a protein, a peptide, or another type of an amino acid sequence.

Antibody is an important type of protein for disease diagnosis and treatment. The structures of antibodies are closely related to their functions, so that antibody structure prediction, which aims to predict the 3D coordinates of atoms in an antibody, is essential in biological and medical applications such as protein engineering, modifying the antigen binding affinity, and identifying an epitope of specific antibody. However, manual experimental methods such as X-ray crystallography are time consuming and expensive.

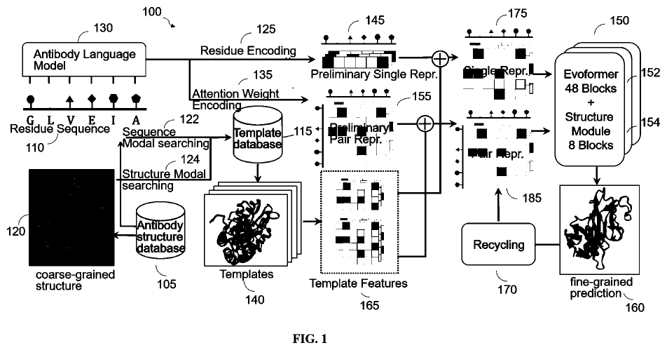

The described techniques provides a computer-implemented solution to predict protein structure, especially antibody structure, based on machine learning or artificial intelligence (AI) technologies. The described techniques include example models, architectures or systems (collectively referred to as “systems”) configured to predict antibody structure from antibody sequences using an antibody Language Model (ALM). One example system is referred to as “xTrimoABFold,” as described in more detail below with respect to (w.r.t.) FIG. 1. Different variants or extensions of xTrimoABFold are also described. For example, one variant is referred to as “xTrimoABFold++” which is described in more detail below w.r.t. FIG. 10.

Conventional protein structure prediction techniques typically rely on MSA to predict a structure of a target protein sequence. MSA refers to the process or the result of sequence alignment of three or more biological sequences. An MSA of an amino acid sequence can include a sequence alignment of an amino acid sequence (e.g., the target antibody sequence) with multiple additional amino acid sequences such as from other homologous proteins, using computational sequence alignment technique, e.g., progressive alignment construction. MSA involves computationally-expensive MSA search.

The described techniques are non-MSA-based or MSA-free protein structure prediction techniques. The described techniques use an ALM, for example, via a transformer model, to learn informative representation of antibodies. The ALM can mine homologous sequence information without complex manual preparation of MSAs. In some embodiments, the described techniques, use the ALM to generate single and pair representations instead of MSAs.

The described techniques can also improve the prediction accuracy compared to MSA-based protein structure prediction techniques. Unlike general proteins, antibodies do not evolve naturally but rather they bind to specific antigens and evolve specifically (fast and one-way evolving). MSAs of antibodies especially on complementarity-determining regions (CDRs) are not always available or reliable, which can hurt the accuracy of models on antibody data.

Moreover, the described techniques employ the pre-trained ALM to extract the information of a single sequence, which performs better than protein structure prediction techniques using general protein language models (PLMs) that are trained on protein databases. In some embodiments, the described techniques train an ALM based on antibody sequences specifically for the antibody applications. For example, the ALM is trained or finetuned on a large-scale Observed Antibody Space (OAS) database. The ALM can learn more specific language information and can perform more powerful representations than general PLM for antibody related downstream tasks.

In some embodiments, for protein structure prediction, template structures may be a kind of auxiliary information to improve the quality of structure models. The described techniques also include computationally efficient template searching algorithms that are designed based on sequence modality and/or structures modality. For example, a cross-modal homologous structure searching algorithm is designed to search templates and provide a good starting point for the antibody structure prediction.

In some embodiments, the described techniques can train an overall model to predict antibody structures in an end-to-end fashion by solving an optimization problem to minimize a loss function. For example, the described techniques can use a structure prediction model that includes an evoformer and structure modules (e.g., similar to those of AlphaFold2) to learn antibody structures in an end-to-end fashion. In some embodiments, the described techniques introduce several forms of loss functions that can provide more accurate prediction results. For example, the described techniques introduce a domain specific focal loss on complementarity-determining regions (CDRs) of antibodies, and/or a differentiable root-mean-squared-deviation (RMSD) loss, in addition to or in place of frame aligned point loss, to better model a difference between a predicted and an accurate structure of an antibody. In some embodiments, one or more of the losses (e.g., the domain specific focal loss on CDRs or RMSD loss) can be used during training and/or fine-tuning of the model. In some embodiments, one or more of the losses (e.g., the domain specific focal loss on CDRs or RMSD loss) are used only during fine-tuning, rather than during training of the model. The described techniques can achieve better prediction performance compared to existing techniques.

In some embodiments, the described techniques can improve the computational efficiency and achieve higher prediction accuracy of antibodies, especially on the CDRs of antibodies. The described techniques can be applied in scenarios, for example, industrially high-throughput drug design, which are not physical or practical for existing techniques. Despite some of the examples are described with respect to antibody structure prediction, which is important in drug discovery, the described techniques can be applied general protein prediction and complex prediction. In some embodiments, compared to existing techniques, the described techniques can improve both accuracy and efficiency in antibody structure prediction, making it a valuable tool for de novo antibody design, and can make further improvement in immuno-theory.

In some embodiments, the described techniques can help better understand antibody structure and its paratope to facilitate a mechanistic understanding of its function. The described techniques can facilitate design of a novel antibody whose paratopes bind to a specific antigen with correct epitopes. In some embodiments, the described techniques can facilitate generating, synthesizing, screening, modifying, or otherwise designing proteins with more accurate and efficient prediction of the structure of the proteins.

The techniques described in this disclosure can generate additional or different technical effects. In some embodiments, the described techniques can be implemented as a software-implemented application or package that can efficiently predict a structure of a target protein. Compared to other computer-assisted protein structure prediction techniques, the described techniques can reduce computational load and improve the computational efficiency. Experiments have been conducted and show that the techniques described outperform AlphaFold2 and other PLM-based SOTAs, e.g., OmegaFold, HelixFold-Single, and IgFold with a large significant margin (30+% improvement on RMSD) while performs 151 times faster than AlphaFold2.

FIG. 1 is a diagram illustrating diagram illustrating an example computer-implemented system 100 configured for protein structure prediction, in accordance with embodiments of this specification. In some embodiments, the example computer-implemented system 100 provides an antibody structure prediction pipeline based on the AlphaFold2 architecture, but without the computationally expensive MSA searching. The example computer-implemented system 100 provides a non-MSA-based or MSA-free protein structure prediction. The example computer-implemented system 100 is referred to as “xTrimoABFold” in this specification.

In some embodiments, the xTrimoABFold 100 takes an amino acid sequence (also referred to as a residue sequence) 110 as input, and generates a fine-grained antibody structural prediction 160 as output.

In some embodiments, xTrimoABFold 100 uses the pre-trained ALM 130 to generate residue encoding 125 and attention weight encoding 135, and uses a transforming result of residue encoding 125 and attention weight encoding 135 to initialize a single representation 175 and a pair representation 185, respectively, which can compensate for the loss of homologous information of MSAs.

In some embodiments, structure templates which model homologous structures of the target antibody can provide a good prior for structure prediction. In some embodiments, xTrimoABFold 100 can additionally use a template searching algorithm to find structure templates 140 based on the sequence of the target antibody and/or the coarse grained prediction structure of the target antibody. xTrimoABFold with template searching can be referred to as xTrimoABFold+Tmpl. Features extracted from the structure templates (referred to as template features) 165 can be incorporated to a transforming result of the residue encoding 125 (preliminary single representation 145) and a transforming result of attention weight encoding (preliminary pair representation 155), resulting in the single representation 175 and the pair representation 185, respectively.

The single representation 175 and the pair representation 185 are fed into a structure prediction model 150 to predict the fine-grained prediction 3D structure 160. In some embodiments, the structure prediction model 150 includes a combination of an encoder and a decoder. As an example shown in FIG. 1, the encoder can be a transformer-based encoder that mixes information between the single representation and pair representation to obtain updated single representation and pair representation. An example of the encoder is an evoformer 152 similar to what is used in AlphaFold2. In some embodiments, the decoder can be a structure module that transforms the abstract representation into concrete 3D atom coordinates. As shown in the example architecture 100, the decoder can be a structure module 154 similar to what is used in AlphaFold2. In some embodiments, the structure prediction model 150 can iteratively update the input of the encoder by recycling the output of the encoder and the output of decoder for further refinement.

For the single representation, a pre-trained ALM (e.g., the ALM 130) generates residue (token) level representations (e.g., residue encoding 125) with a single sequence as input (e.g., the residue sequence 110). The residue level representations can be used as an initial value of the single representation 175 of the following encoder (e.g., evoformer 152) by proper transformation.

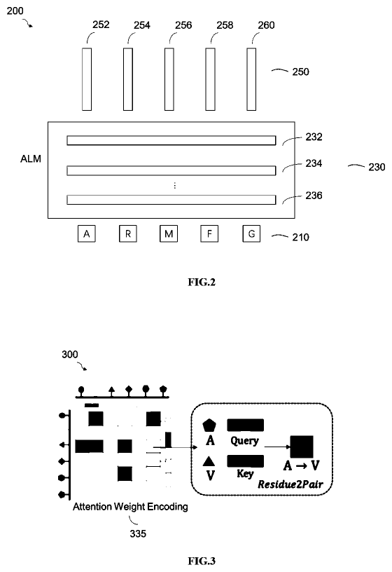

FIG. 2 is a diagram 200 of an example input 210 and output 250 of an ALM 230 in an example computer-implemented system configured for antibody structure prediction (e.g., the xTrimoABFold 100), in accordance with embodiments of this specification. In some embodiments, the ALM 230 can be an example implementation of the ALM 130, or another computer-implemented system configured for antibody structure prediction. In some embodiments, the ALM 230 can be a deep machine learning model that includes multiple neural network blocks such as blocks 232, 234, and 236. In some embodiments, each block of the ALM 230 can be a self-attention network that includes one or more self-attention layers.

With an input x, an output z of an ALM can be represented as follows:

$$z = ALM ( x ) , z ∈ {R}^{N \times {d}_{lm}} \qquad ( 1 - 1 )$$

where x={x₁, x₂, . . . , x_{N}} denotes the sequence of residues (e.g., the residue sequence 110), N refers to the number of residues in the given protein, dₗₘ is the hidden size of the ALM, and ALM represents the pre-trained ALM.

In some embodiments, the residue sequence can be a sequence of amino acid type identifiers (IDs) (e.g., represented by letters A, R, M, F, G, etc.). Each amino acid can correspond to a dₗₘ-dimension embedding, for example, based on one-hot encoding. As such, N amino acids correspond to a N×dₗₘ embedding. In this case, before the ALM, there can be an embedding layer that maps an amino acid type ID into a dₗₘ-dimension embedding (e.g., a 1×dₗₘ vector), and the input x to the ALM in Equation (1-1) can be an embedding that has a size of N×dₗₘ.

In some other embodiments, the input x to the ALM can be a sequence of amino acid type IDs that has a size of N×1. The ALM can include, as a first layer of the ALM, an embedding layer that maps an amino acid type ID into a dₗₘ-dimension embedding. The ALM can include other layers such as self-attention layers to update the embedding output from the first layer.

Given the residue sequence 110 as an example of the residue sequence of a protein.x, the output z of the ALM can be an example of the residue encoding 125.

The output z of the ALM can be used to compute a preliminary single representation (e.g., the preliminary single representation 145) as follows:

$${s}^{0} = Linear ( z ) , {s}^{0} ∈ {R}^{N \times ds} \qquad ( 1 - 2 )$$

where s⁰ is the preliminary single representation, dₛ is the hidden size of the following encoder (e.g., the evoformer 152) corresponding to the single representation, and Linear refers to the linear layer of a neural network (e.g., a fully convolutional neural network (FCNN)) that is used to transform the output z into the preliminary single representation. In some embodiments, structure templates are not employed in the structure prediction, s⁰ can be used as the initial single representation of the following encoder directly; in some embodiments, structure templates are employed in the structure prediction, and s⁰ can be incorporated with template features to obtain the initial single representation.

In some embodiments, the input 210 of the ALM 230 can be a sequence of tokens. In some embodiments, the input 210 can be an amino acid sequence or a residue sequence that includes multiple amino acids or residues, such as the residue sequence 110. As an example shown in FIG. 2, the input 210 includes N=5 residues, namely, x={A, R, M, F, G} in this case. Each of the residues can be regarded as a token, and the ALM 230 can generate an embedding corresponding to each of the residues in the residue sequence 210. In the example shown in FIG. 2, the output 250 z of the ALM 230 includes 5 embeddings 252, 254, 256, 258 and 260 corresponding to each of the 5 residues, A, R, M, F, and G. In this example, each embedding can have a dimension of 1×dₗₘ, and the output z 250 has a dimension of 5×dₗₘ.

In some embodiments, the ALM 230 adopts the mechanism of multi-head self-attention, and each token can get information from other tokens, which can be seen as a residue2pair communication. For the pair representation, the attention weights of the multi-head self-attention mechanism in the ALM are rich in prior knowledge about the relation between residues such as position information, which can be combined as the preliminary single representation 155 through adaptive transformation.

As an example, the ALM can have a multi-head self-attention structure (e.g., an ALM with L attention layers and each layer with H attention heads). The h-th attention head in the 1-th layer has learnable parameters

$${W}_{q}^{h , l} , {W}_{k}^{h , l} , {W}_{v}^{h , l} ,$$

which represents learnable parameters correspond to querys, keys and values of the self-attention neural network (i.e., the ALM in this example). In some embodiments, each residue can be represented by a respective embedding. For each attention head in each layer, an embedding corresponding to a residue of the input residue sequence 110 can serve at least two roles, a query and a key, to update its own embedding as well as help updating another residue's embedding. For example, an input into the 1-th multi head attention layer of the ALM can be an embedding x¹ (including

$${x}_{1}^{l} , {x}_{2}^{l} , {x}_{3}^{l} , {x}_{4}^{l} … {x}_{N}^{l} ) ,$$

where

$${x}_{i}^{l}$$

corresponds to the embedding of residue i of the residue sequence of N residues. The 1-th multi head attention layer with H attention heads of the ALM can process x¹ and obtain x^{out}, and x^{out} can be directly used as or transformed to x^{l+1} (including

$${x}_{1}^{l + 1} , {x}_{2}^{l + 1} , {x}_{3}^{l + 1} , {x}_{4}^{l + 1} … {x}_{N}^{l + 1} )$$

that can be input into the (1+1)-th multi head attention layer of the ALM.

In some embodiments, the generation of the preliminary pair representation p⁰ using the ALM can be formalized as follows:

$${Q}_{i}^{h , l} = {W}_{q}^{h , l} {x}_{i}^{l} , \qquad ( 2 - 1 )$$

$${K}_{j}^{h , l} = {W}_{k}^{h , l} {x}_{j}^{l} , \qquad ( 2 - 2 )$$

$${B}_{ij}^{h , l} = \frac{{{Q}_{i}^{h , l} ( {K}_{j}^{h , l} )}^{T} + {a}_{ij}}{\sqrt{{d}_{lm}}} , \qquad ( 2 - 3 )$$

$${A}^{h , l} = softamx ( {B}^{h , l} ) , \qquad ( 2 - 4 )$$

$${q}_{ij} = Concat ( {A}_{ij}^{h , l} \vert l ∈ [ 1 , L ] , h ∈ [ 1 , H ] ) , \qquad ( 2 - 4 )$$

$${p}^{0} = Linear ( q ) , \qquad ( 2 - 6 )$$

where Qᵢ^{h,l} and Kⱼ^{h,l} represent the query and key vectors/embeddings of residues i and j in the l-th layer and k-th head respectively, aᵢⱼ denotes the relative position encoding between the residue i and the residue j (e.g., aᵢⱼ can represent the relative positions of the residue i and the residue j in the residue sequence, which can be a learnable embedding), A^{h,l} represents the attention weight matrix obtained by the h-th attention head in the 1-th layer,

$${A}_{ij}^{h , l}$$

represents the (i,j)-th element of the matrix A^{h,l},

$${B}_{ij}^{h , l}$$

represents the (i,j)-th element of the matrix B^{h,l}, qᵢⱼ represents the (i,j)-th element of the matrix q∈R^{N×N×HL}, p⁰∈R^{N×Ndp} and dₚ is the hidden size of the encoder corresponding to the single representation.

In addition, the 1-th layer has another learnable parameter Wₒ^{l}, which can be used to generate x^{out}, for example, as follows:

$${x}^{out} = {W}_{o}^{l} {x}^{out ’} , \qquad ( 2 - 7 )$$

wherein x^{out′} can be obtained by V^{1,l} A^{1,l}, V^{2,l} A^{2,l}, . . . , V^{H,l} A^{H,l}, for example, by concatenation, wherein:

$${V}_{i}^{h , l} = {W}_{v}^{h , l} {x}_{i}^{l} . \qquad ( 2 - 8 )$$

x^{out} can be directly used as or transformed to x^{l+1}. In some embodiments, the transformation includes, for example, normalization and/or feed forward.

The above calculation can be regarded as an example residue2pair communication because of multi-head query-key product of residue pairs are involved in this step. For example, given a pair of amino acid residues i and j of the input residue sequence 110, a multi-head query-key product

$${{Q}_{i}^{h , l} ( {K}_{j}^{h , l} )}^{T}$$

is calculated. As an example, if the ALM has L=10 layers and each layer has H=3 attention heads, qᵢⱼ can be a vector of size HL=30, wherein the first 3 elements (e.g., element 0-2) of qᵢⱼ correspond to attention weights of the 3 attention heads of the first layer, the second 3 elements (e.g., element 3-5) of qᵢⱼ correspond to attention weights of the 3 attention heads of the second layer, and so on. In some embodiments, qᵢⱼ can include attention weights of the H attention heads of the L layers concatenated, collected, or assembled in another manner.

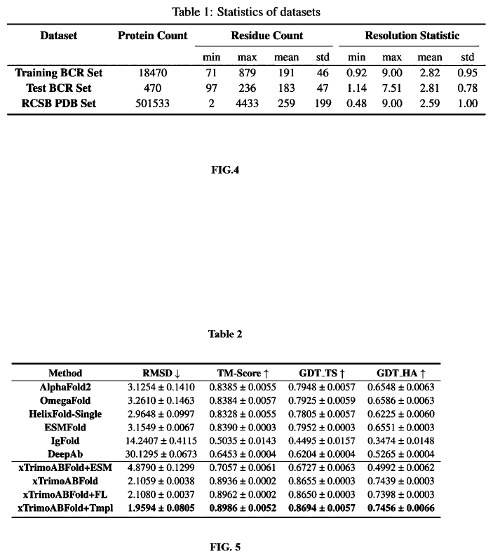

FIG. 3 is a diagram of an example illustration 300 of residue2pair communication in an example computer-implemented system (e.g., the xTrimoABFold 100) configured for protein structure prediction, in accordance with embodiments of this specification. In this example illustration 300, the attentions weight encoding 335 (e.g., the attention weight encoding 135) of the multi-head self-attention mechanism in the ALM can include a second embedding (e.g., qᵢⱼ as shown in Equation (2-4)) obtained when an amino acid residue A (e.g., residue i) is used as a query and an amino acid residue V (e.g., residue j) is used as a key in the multi-head self-attention mechanism.

In some embodiments, structure templates may provide a good prior for structure prediction. Unlike previous works such as AlphaFold2 that search templates by MSAs-based algorithms (e.g., HHSearch that detects templates by Hidden Markov Model (HMM)-HMM alignments between query and target database), a MSA-free template searching algorithm is introduced in this disclosure. The template searching algorithm does not depend on MSAs and can be memory- and computation-efficient. In some embodiments, the template searching algorithm can be a cross-modal homologous searching algorithm that introduces two perspectives, sequence and structure, to search templates without MSAs.

For example, xTrimoABFold+Tmpl adopts a cross-modal template searching algorithm that search homologous structures in both sequential and structural modals. The cross-modal template searching algorithm that includes both a sequence modal searching (also referred to as a sequential modal search) 122 and a structural modal searching 124. The sequence modal searching 122 searches for one or more structures of one or more sequences that are similar to the input amino acid sequence 110 in the template database. For example, a coarse-grained structure 120 can be used as part of the input when using structural modal searching 124. The structural model searching 124 searches for one or more structures that are similar to the input coarse-grained structure 120 in the template database. The template database used in the a sequence modal searching 122 and the Structural model searching 124 can be the same database or different databases. In some embodiments, xTrimoABFold+Tmpl can use a single modal template searching.

In some embodiments, the template searching algorithm can be conducted in a protein structure database or an antibody database. In some embodiments, before conducting template search, a protein structure database and/or an antibody database can be constructed, which can be used as a structure template database.

For the sequence modal searching 122, taking into account the idea that similar antibody sequences are likely to have similar 3D structures, a similarity score or an alignment score such as a sequence alignment based similarity score can be used to search the structures of sequences similar to the target antibody sequence from the template database as the templates. An example similarity score function is formalized as:

$$Sim ( {x}_{1} , {x}_{2} ) = Align ( {x}_{1} , {x}_{2} ) ) / max ( len ( {x}_{1} ) , len ( {x}_{2} ) ) ,$$

where x1 and x2 are residue sequences, and Align (.,.) is the sequence alignment, which denotes the maximum matched residues between two amino acid sequences (e.g., Align(‘GVI’,‘GIV’)=2). Various existing algorithms (e.g., the Needleman-Wunsch algorithm) can be used for sequence alignment computation. Additional or different formula or algorithms can be used as the similarity score or be used to calculate the similarity score.

In some embodiments, the sequential modal searching first screens out all sequences whose similarity scores are within a range, such as in the range of (0.4, 0.95), and restricts the available templates up to a certain number, Tₛₑ, (e.g., Tₛₑ=10) with the maximum similarity scores to the target antibody sequence. After that, the structures corresponding to these top Tₛₑ sequences will be considered as part of template candidates for the following training or inference.

In some embodiments, in terms of the efficiency of the search algorithms, sequential modal searching is more efficient than MSA-based algorithms. The sequential modal searching can provide both real-time searching and batch searching. In some embodiments, real-time searching can search the templates of the target sequence within 1s through a parallel search algorithm. In some embodiments, real-time searching divides the template database into N_{workers} parts and implements parallel searching to select N_{workers}*Tₛₑ candidates, and then sorts the searched candidates with the similarity scores through merge sort. Since the merge sort is a stable algorithm, the same results can be guaranteed for each real-time search. Finally, the top Tₛₑ of the sorted homologous structures are selected as templates. In some embodiments, batch searching can compress the time cost for a single sequence of template search to the level of milliseconds by parallel search and storage of a large number of sequences.

Structural modal searching 124 focuses on finding similar structures in a database based on the coarse-grained structure 120 of the target antibody even though the sequences of these structures may not match the target antibody. The coarse-grained structure 120 can be an estimated, predicted, or otherwise obtained structure that is used as an initial or baseline structure template to search for similar structures. In some embodiments, the coarse-grained structure 120 can be configured as a default structure (e.g., based on knowledge of a structure that is similar to that of the target antibody, or that provides a good starting point for the target antibody). In some embodiments, the coarse-grained structure 120 can be a structure prediction obtained from another structure prediction algorithm or model based on the sequence of the target antibody.

Structural modal searching 124 can use the same or different similar score compared to the sequential modal searching 122. In some embodiments, similar to the sequential modal searching 122, similarity scores between the coarse-grained structure of the target antibody and structures in a template database (e.g., template database 115) are computed. Various existing algorithms or tools (e.g., FoldSeek tool) suitable for structure-pairwise alignment can be used to calculate the alignment scores. The structural modal searching 124 can determine up to a certain number, Tₛₜ, (e.g., Tₛₜ=10) of structures with top similarity scores. In some embodiments, the structures with too high similarity (e.g., larger than 0.95 or another threshold) are removed to exclude the target antibody itself. The resulting top Tₛₜ structures can be added to the template candidate set.

After the cross-modal template searching, a total number of T template candidates can be obtained. In some embodiments, T is less than or equal to Tₛₑ. Tₛₜ because of potential duplication of two modal search results. The values of T, Tₛₑ, and Tₛₜ can be configured. For example, in a case where T=4, Tₛₑ=2 and Tₛₜ=2, 4 templates can be chosen from a candidate set of top-2 sequential modal templates and top-2 structural modal templates at inference time. In some embodiments, in the training step, a number (e.g., min (Uniform [0, 7], S)) of templates can be randomly selected out of this restricted set of T templates, where S can be configured as well. For example, S=4. In some embodiments, the structures selected by two searching algorithms contain more homologous structure information, so a higher sampling probability can be assigned to these structures.

In some embodiments, features extracted from the structure templates (referred to as template features 165) can be incorporated to a preliminary single representation 145 that is a transforming result of the residue encoding 125 and a preliminary pair representation 155 that is a transforming result of the attention weight encoding 135, resulting in the single representation 175 and the pair representation 185, respectively. For example, an template encoder (e.g., the template encoder of AlphaFold2) can be used to encode the template structures into two types of template features, template angle features and template pair features. And the template angle features and template pair features are incorporated to the preliminary single and pair representations respectively, which can be formalized as follows:

$${s}^{\hat{} 0} = Concat ( {s}^{0} , {f}_{ta} ) , \qquad ( 4 - 1 )$$

$${p}^{\hat{} 0} = {p}^{0} + {f}_{tp} ; \qquad ( 4 - 2 )$$

where fₜₐ∈R^{T×N×ds}, s\hat{ }∈R^{(T+1)×N×ds}, p\hat{ }⁰, fₜₚ∈R^{N×N×dp}, fₜₐ and fₜₚ are the template angle and pair features respectively, and s\hat{ }⁰ and p\hat{ }⁰ are the single and pair representations with template features, T is the number of templates. In some embodiments, fₜₐ and fₜₚ can be exacted using methods similar to those of AlphaFold2. For example, fₜₐ can be constructed by concatenating: template_aatype, template_torsion_angles, template_alt_torsion_angles, and template_torsion_angles_mask. fₜₚ can include concatenation of the pair residue features template_distogram, template_unit_vector, and also several residue features, which are transformed into pair features.

s\hat{ }⁰ and p\hat{ }⁰ can be taken as the input of the encoder of the structure prediction model 150. In some embodiments, the evoformer 152 of AlphaFold2 can be used as the encoder to model complex information in initial single and pair representations. Note that the column-wise gated self-attention of evoformer 152 can exchange the sequence information modeled by the ALM 130 with the structure information of templates 140. The structure module 154 can employ several geometric transformation operators such as Invariant Point Attention (IPA) to predict the 3D structures of the protein end-to-end. In this example, the evoformer 152 includes 48 blocks and the structure module 154 includes 8 blocks. In some other embodiments, the evoformer and the structure module can include a different number of blocks. For example, when the embedding predicted by the ALM is good, the number of blocks in the evoformer can be less, such as 1 block. Moreover, a recycling mechanism 170 is employed to refine the predicted structures 160 iteratively.

In some embodiments, xTrimoABFold 100 is trained end-to-end to optimize an objective function or minimize a loss function. Compared to the loss function used by AlphaFold2 that incudes framed aligned point error (FAPE) and a number of auxiliary losses, the loss function of xTrimoABFold 100, a non-MSA-based or MSA-free structure perdition system, removes the loss on masked MSA.

In some embodiments, the loss function used by xTrimoABFold 100 can be formalized as follows:

$${L}_{train} = 0.5 {L}_{FAPE} + 0.5 {L}_{aux} + 0.3 {L}_{dist} + 0.01 {L}_{conf} , \qquad ( 5 )$$

where L_{FAPE} refers to the FAPE overall atoms in the amino acid sequence, Lₐᵤₓ are the averaged FAPE and torsion losses on the inter-mediate structures over Cₐ only, L_{dist} is an averaged cross-entropy loss for distogram prediction, and L_{conf} is the model confidence loss. These losses can be computed, for example, according to existing methods such as those disclosed in AlphaFold2.

In some embodiments, the loss function of xTrimoABFold 100 can include other loss/error/distance metrics. For example, since the structure of complementarity determining region (CDR) in antibody is usually hard to predict than other framework regions (FR), the loss function can further include a CDR focal loss. In some embodiments, the CDR focal loss can be used in both training and fine-tuning xTrimoABFold. In some embodiments, the CDR focal loss can be used only to fine-tune xTrimoABFold after training the xTrimoABFold with a loss function without the CDR focal loss. In some embodiments, such a variant of xTrimoABFold of using the CDR focal loss for fine-tuning but not during training is referred to as xTrimoABFold-FL (focal loss). In one example, the CDR focal loss is denoted as:

$${x}_{ij} = {{T}_{j}^{- 1}}_{0} {x}_{i} , {x}_{ij}^{true} = {{T}_{j}^{{true}^{- 1}}}_{0} {x}_{j}^{true} , {T}_{j} , {T}_{j}^{true} ∈ ( {ℝ}^{3 \times 3} , {R}^{3} ) , {x}_{i} , {x}_{i}^{true} ∈ {ℝ}^{3} . \qquad ( 6 )$$

$${d}_{ij} = \sqrt{{‖ {x}_{ij - {x}_{ij}^{true}} ‖}^{2} + ϵ ,} , ϵ = {10}^{- 4} {Å}^{2} \qquad ( 7 )$$

$${ℒ}_{{fc}_{CDR}} = \frac{1}{Z} \frac{1}{{N}_{atoms}^{CDR}} {\sum }_{i ∈ \{ 1 , … , {N}_{atoms}^{CDR} \}} \frac{1}{{N}_{frame}} {\sum }_{j ∈ \{ 1 , … , {N}_{frame} \}} min ( {d}_{clamp} , {d}_{ij} ) , {d}_{clamp} , Z = 10 Å \qquad ( 8 )$$

$${ℒ}_{fine - tune} = {ℒ}_{train} + {λℒ}_{{fc}_{CDR}} \qquad ( 9 )$$

where xᵢ, and x^{true}ᵢ are the prediction and ground-truth 3D coordinates of atom i in CDR regions respectively, Tⱼ and

$${T}_{j}^{true}$$

represent the SE(3) transformations, which are calculated based on xᵢ, and xₜᵣᵤₑ respectively and include rotation ( [symbol] ^{3×3}) and translation ( [symbol] ³), ° represent Hadamard product, Nₐₜₒₘₛ^{CDR} denotes the number of atoms in CDR regions of antibodies, and N_{frames} is the number of local frames. Fine-tuning with L_{fine-tune} helps xTrimoABFold pay more attention to the difficult CDR regions. In this example, both d_{clamp} and Z are set to be 10 Å, which means that if dᵢⱼ is larger than 10 Å, dᵢⱼ is set to be 10 Å because any larger distance is considered not beneficial for the prediction. In some embodiments, d_{clamp} and Z can be set at other values to improve the prediction performance.

In some embodiments, the loss function can further include a RMSD loss in addition to or in place of the FAPE loss (and/or other losses). The RMSD loss can be a more accurate measure because the FAPE loss is an upper bound of RMSD. In some embodiments, a differentiable RMSD loss is developed to improve the prediction accuracy:

$${L}_{{rmsd}_{ca}} = \sqrt{\frac{1}{{N}_{atom}}} {\sum }_{i} {( {x}_{i}^{pred} - {T}^{align} . {x}_{i}^{gt} )}^{2} , \qquad ( 10 )$$

where Nₐₜₒₘ is the number of atoms, xᵢ^{pred}, xᵢ^{gt} and are the prediction and ground-truth 3D coordinates, and T_{align} is a SE(3) transformation for them. Compared to the FAPE loss which has a transformation on a frame level, here T^{align} can be on a global level of the entire amino acid sequence.

In some embodiments, one or more protein structure databases can be collected, created, downloaded, received, or otherwise obtained, for example, for template searching, and/or for training the ALM, and/or other components of a computer-implemented system configured for protein structure prediction (e.g., the xTrimoABFold 100 or xTrimoABFold++ 1000). In an experiment, two large datasets are created. The first one is the 19K antibody structure dataset 105 as shown in FIG. 1. A total of 18937 antibody data are obtained, which include both amino acid sequences and structures selected from RCSB Protein Data Bank (PDB) released before Apr. 13, 2022. The specific selections focusing on the structures and sequences are as follows. First, each PDB file is split into single chains, and then the selection is made. On one hand, among the whole 19736 BCR chains from PDB, samples that have no structure resolution values or those of which the structure resolution is larger than 9 Å were filtered out to keep the quality of structure data. On the other hand, as for the sequences, we filtered out the samples whose sequence is empty or whose repetition rate of a kind of amino acid is more than 90 percent in a sequence is filtered out. Besides, deduplication are also conducted on the sequence and the samples that have lower structure resolution are kept. After these filtering processes, 18937 antibody data are obtained as the antibody structure dataset 105. Among these, data released before Jan. 17, 2022 that contains 18470 samples are used as the training set, while the other 470 samples are used as the test set in one example implementation.

In some embodiments, the antibody structure dataset 105 is used as the training dataset of xTrimoABFold (and its variants). In the training stage, antibody data (including an antibody sequence and corresponding actual structure) of a training antibody can be selected from the antibody structure dataset 105 to obtain its coarse-grained structure, and to determine the template candidates through sequence searching using the antibody sequence and/or structural modal searching described above based on the coarse-grained structure. T templates from template candidates can be selected after the template search. The structure of the training antibody can be predicted based on the antibody sequence and the templates of the training antibody using an initial xTrimoABFold (e.g., an untrained with initial model parameters, or a model whose parameters have been updated for several training iterations, but have not been fully trained). The loss between the predicted structure and the actual structure of the training antibody can be calculated, for example, based on the techniques described in this disclosure. The model parameters of xTrimoABFold are then updated based on the loss. The above process can be repeated for other antibody data of other training antibodies in the training database.

The second dataset is the 501K protein structure database. The whole protein database can be downloaded from RCSB PDB. A total of 593491 protein chains can be obtained after filtering out the missing structure file. Later, the parts out of specification on structure resolution and sequence similarity are removed as mentioned above. Repeated examples are removed as well. In the end, the 501K protein structure database is obtained, which includes a total of 501533 protein chains. The protein structure database can be used as the template database, e.g., template database 115, for template search.

FIG. 4 includes Table 1 illustrating statistics of example datasets of the 19K antibody structure dataset 105 and the template database 115 that includes 501K protein structures, in accordance with embodiments of this specification.

The xTrimoABFold method is compared with several latest state-of-art protein structure prediction methods: AlphaFold2, OmegaFold, PLM-based HelixFold-Single, ESMFold, ALM-based IgFold, and DeepAb, which are used as baselines for comparison. For AlphaFold2, the inference is made using five different models and picked up the structures with the highest predicted local distance difference test (pLDDT) confidence for benchmarking. In some experiments, a variant of the xTrimoABFold model, referred to as xTrimoABFold-ESM, is trained. The xTrimoABFold-ESM replaces the ALM with a general protein language model of ESM2. The performance of xTrimoABFold-ESM is worse than xTrimoABFold, which demonstrates that the ALM is a better option than general protein language model.

To evaluate the quality of antibody structure prediction, root-mean-squared-deviation (RMSD), TM-Score, GDT TS and GDT HA can be used as the evaluation metric. Both two values can be calculated over backbone heavy atoms after alignment of the respective framework residues by DeepAlign. In order to evaluate the performance of CDR loops which are considered difficult for a model to predict, 3 CDR regions of antibody structure are extracted and these regions are evaluated based on the local and global alignments respectively. On the scheme of local alignment, two local CDR regions are aligned and RMSD is calculated on the local alignment matrix. On the scheme of global alignment, two complete antibody structures are used to generate the alignment matrix, and RMSD is computed based on this alignment matrix.

In some embodiments, the TM-score can be computed as follows:

$$TM - Score = max [ \frac{1}{{L}_{target}} {\sum }_{i}^{{L}_{common}} \frac{1}{1 + ( \frac{{d}_{i}}{{{d}_{0} ( ( \frac{{d}_{i}}{{L}_{target}} ) )}^{2}} )} ]$$

where L_{target} is the sequence length of target protein and L_{common} is the number of residues that appear in both the template and target structures.

In one example experiment, for the ALM 130, AntiBERTy (Version 0.0.5, installed from PyPI), a BERT-based pre-trained protein language model, trained on OAS with 558M antibody natural sequences is used to generate residue-level representations. The hidden dimension of the ALM is 512 and the feedforward dimension is 2048. AntiBERTy contains 8 layers, with 8 attention heads per layer. In total, AntiBERTy contains approximately 26M trainable parameters. In some embodiments, in the training phase, the gradient backpropagation of the ALM can be blocked, and only the evoformer 152 and the structure module 154 are trained. In some embodiments, the Adam Optimizer with the learning rate of 1e-3, β₁=0.9, β₂=0.999, ϵ=8 and weight decay of 0 can be used for the training. In some embodiments, the gradient can be clipped using the threshold of 10e9. In the example experiment, the model was trained for 25 epochs in 46 hours on 8 NVIDIA A100 GPUs with a stayed batch size of 8. Similar to AlphaFold2, the crop size of the sequence is set to 256. On account of the replacing of MSA representation with the single sequence representation of ALM, InputEmbedder, ExtraMSAEmbedder and ExtraMSAStack, as well as the masked MSA loss are removed, compared to AlphaFold2. When making structural modal searching, Foldseek which enables fast and sensitive comparisons of large structure sets was used. 3Di Gotoh-Smith-Waterman is chosen as the alignment type and max-seq is set to 2000.

The results of main experiments that compare xTrimoABFold with the baselines contain two parts: one is the model performance on evaluation metrics, and the other is for the time efficiency. Tables 2, 3 and 4 in FIGS. 5 and 6 respectively show the accuracy performance of models on antibody structure prediction and CDR loop structure prediction. For brevity, only RMSD and TM-score for three CDR loops are presented. Specifically, Table 2 shows experimental results of antibody structure prediction on test dataset with 95% confidence interval. xTrimoABFold-ESM refers to a similar approach to xTrimoABFold except for replacing the pre-trained ALM with the pre-trained PLM, ESM2, with 15b parameters (the largest PLM to date). The results show ALM is more suitable for antibody structure prediction.

As for the protein structure prediction of CDR loops, which are well-known as difficult domains for a model to make an accurate prediction, xTrimoABFold also performs well. Table 3 and 4 in FIG. 6 show the RMSD of all models based on the local alignment and global alignment respectively. Specifically, Table 3 shows experimental results of antibody CDR loop structure prediction on the local alignment on test dataset with 95% confidence interval. Table 4 shows experimental results of antibody CDR loop structure prediction on the global alignment on test dataset with 95% confidence interval. As shown, xTrimoABFold has improvements over HelixFold-Single and IgFold, which are trained based on a large-scale protein language model and ALM on CDR1 and CDR2 loop. xTrimoABFold yields the best performance in the CDR3 loop which has been proven a difficult domain to predict because of the highly variable and conformationally diverse.

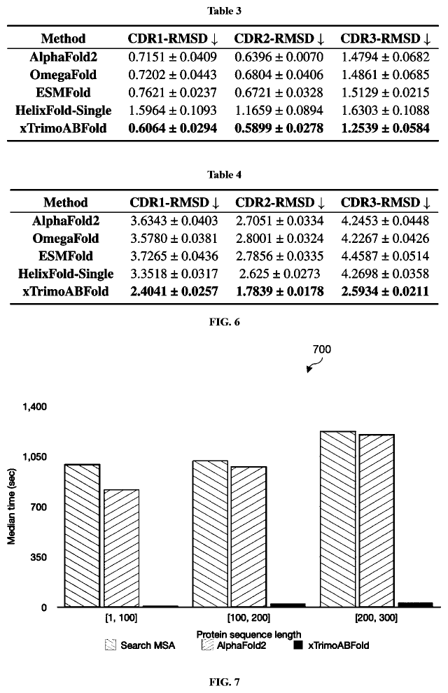

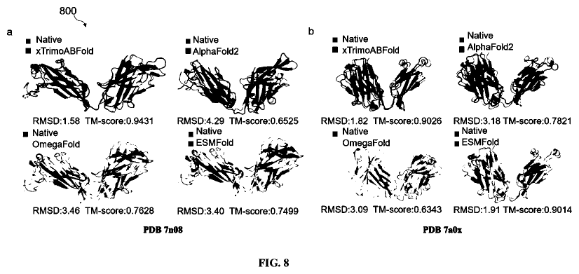

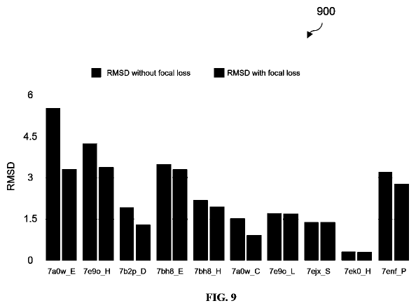

FIG. 7 is a graph 700 illustrating an example experiment result with respect to antibody structure prediction time of different methods on different lengths of amino acid sequence from the test dataset. Specifically, FIG. 7 shows median time of MSA search, AlphaFold2 and xTrimoABFold. AlphaFold2 makes protein structure prediction according to MSAs, which results in massive time consumption. Compared with AlphaFold2, xTiomoABFold is an MSA-free model which predicts the protein structure by a single amino acid sequence with ALM. As shown in FIG. 7, xTrimoABFold is 151 times faster than AlphaFold2, which shows that xTrimoABFold can overcome the bottleneck of time efficiency in protein structure prediction, and enable large-scale antibody structures prediction at a fast speed. xTrimoABFold achieves better time efficiency on structure prediction compared to baselines and can perform a fast antibody structure prediction.

In terms of performance on antibody structure prediction, xTrimoABFold significantly outperforms all baselines on the test dataset. In terms of RMSD, xTrimoABFold makes 37.20%, 40.06%, 34.08%, 38.05%, 86.28%, 93.52% improvements over AlphaFold2, OmegaFold, HelixFold-Single, ESMFold, IgFold, and DeepAb as shown in Table 2. In the meanwhile, this trend continues on other evaluation metrics. xTrimoABFold achieves state-of-art performance on the antibody structure prediction compared with not only PLM-based but also MSA-based protein structure prediction methods.

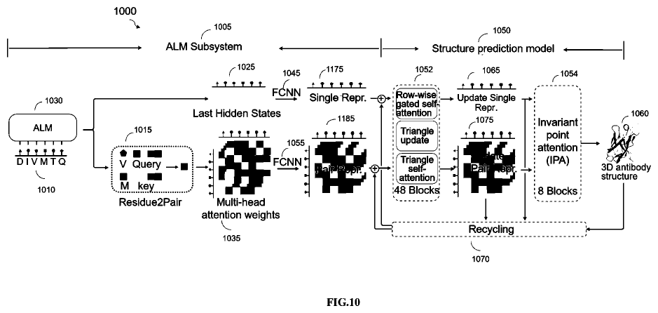

FIG. 8 is a plot 800 illustrating examples of protein structures predicted by xTrimoABFold and other baselines, in accordance with embodiments of this specification. As shown, xTrimoABFold outperforms other baselines including AlphaFold2, OmegaFold, and ESMFold in terms of prediction accuracy.

In the experiment, ablation studies are conducted to evaluate the performance improvement brought by the introduction of pre-trained ALM (e.g., based on AntiBERTy model) and the added CDR focal loss when fine-tuning the model for xTrimoABFold.

xTrimoABFold used a pre-trained ALM (e.g., an AntiBERTy-based model) to generate residue-level representations, which contains more specific antibody information compared to general protein language models like OmegaPLM, ESM-2, etc. In the example ablation study, a variant of xTrimoABFold, xTrimoABFold-ESM, is used to validate the choice of ALM rather than the regular protein language model. xTrimoABFold-ESM replaces the ALM with ESM-2, a largescale protein language model trained on 250 million protein sequences while keeping other parts of xTrimoABFold the same. In the experiment, xTrimoABFold-ESM was trained on the same set of data as xTrimoABFold and got worse prediction performance compared to xTrimoABFold as shown in Table 2, which shows the performance gains from pre-trained ALM in xTrimoABFold.

In order to prove the effectiveness of focal loss, ablation study is performed on another variant of xTrimoABFold, xTrimoABFold+FL. xTrimoABFold+FL adds focal loss into the loss function of xTrimoABFold for fine-tuning as discussed above. The performance of xTrimoABFold+FL is also shown in Table 2. The experiments found that the designed focal loss could effectively improve the performance and reduce the variance.

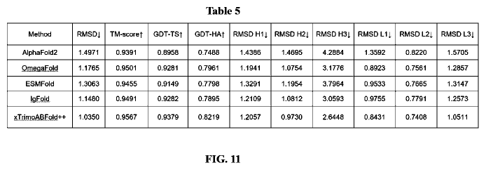

Moreover, in another experiment, ten samples were randomly selected from the test dataset and performance of xTrimoABFold before and after adding CDR focal loss were compared. FIG. 9 is a graph 900 illustrating an example experiment result with respect to antibody structure prediction performance of xTrimoABFold with and without focal loss. In these examples shown in FIG. 9, compared to xTrimoABFold without CDR focal loss, xTrimoABFold with CDR focal loss (e.g., xTrimoABFold+FL) achieves various degrees of decrease of RMSD value of the predicted structures to the ground truth. The performance gains from CDR focal loss shows the focal loss is effective in the antibody structure prediction, especially for the CDR loops which seems difficult to predict for regular models.

Another ablation experiment was also conducted to show the effectiveness of the templates searched by the cross-modal homologous structure searching. Another variant of the xTrimoABFold model, referred to as xTrimoABFold+Tmpl, is used. xTrimoABFold+Tmpl incorporates the cross-modal homologous structure searching into xTrimoABFold and adds the template features 140 into the single representation 175 and the pair representation 185. Table 2 shows the performance of xTrimoABFold+Tmpl, which shows improved predication accuracy compared to xTrimoABFold. The experiment result of xTrimoABFold+Tmpl demonstrates that the templates searched by the cross-modal homologous structure searching can effectively reduce the variance and improve the prediction accuracy.

FIG. 10 is a diagram illustrating diagram illustrating another example computer-implemented system 1000 configured for protein structure prediction, in accordance with embodiments of this specification. The example computer-implemented system 1000 provides a non-MSA-based or MSA-free protein structure prediction. The example computer-implemented system 1000 can be considered as another variant of xTrimoABFold 100 of FIG. 1. The example computer-implemented system 1000 is referred to as “xTrimoABFold++” in this specification. Compared to xTrimoABFold 100 of FIG. 1, xTrimoABFold++ 1000 does not need to perform template search, which further reduces the computational complexity.

In some embodiments, xTrimoABFold++ 1000 takes an amino acid sequence (also referred to as a residue sequence) 1010 as input and generates a fine-grained structural prediction 1060 as output. xTrimoABFold++ 1000 can include two subsystems, an ALM subsystem 1005 and a structure prediction model 1050.

The ALM subsystem 1005 uses a pre-trained ALM 1030 to model homologous antibody sequences and to learn an antibody's representation, e.g., a single presentation, without expensive MSA searching. The ALM 1030 can be the similar to the ALM 130 or 230 described w.r.t. FIG. 1 or 2. The ALM 1030 receives an input amino acid sequence 1010 and outputs last hidden states 1025 of the ALM 1030. In some embodiments, the last hidden states 1025 can be represented as a vector, a matrix, a tensor, or another embedding. The last hidden states 1025 can be transformed into a single representation 1175, for example, via a fully convolutional neural network (FCNN) 1045 or another method, such that the single representation 1175 has a proper dimension to be input to a following structure prediction model 1050 (e.g., an input to an encoder 1052 of the structure prediction model 1050). Using the example described w.r.t. Equations (1-1) and (1-2) and FIG. 2, the last hidden states 1025 can have a dimension of N×dₗₘ, and the FCNN 1045 is used to transform the last hidden states 1025 to the single representation that has a dimension of N×dₛ, if the hidden size of the encoder 1052 is dₛ.

ALM 1030 can also be used to obtain a pair presentation 1185 to be input into the following structure prediction model 1050. In some embodiments, a residue2pair communication 1015 can be used to obtain multi-head attention weights 1035, for example, according to the example techniques described above w.r.t. Equations (2-1)-(2-8) and FIG. 3 or another technique. The multi-head attention weights 1035 can be transformed into a pair representation 1185, for example, via another fully convolutional neural network (FCNN) 1055 or another method, such that the pair representation 1185 has a proper dimension to be input to a following structure prediction model 1050 (e.g., an input to the encoder 1052 of the structure prediction model 1050). Using the example described w.r.t. Equations (2-1)-(2-8) and FIG. 3, the multi-head attention weights 1035 can have a dimension of N×N*HL, and the FCNN 1045 is used to transform the multi-head attention weights 1035 to the pair representation that has a dimension of N×N×dp.

The structure prediction model 1050 can be the same as or different from the structure prediction model 150 of FIG. 1. In some embodiments, the structure prediction model 1050 has a deep learning architecture. In some embodiments, the structure prediction model 1050 includes a combination of an encoder 1052 (e.g., evoformer in Alphafold2) and decoder 1054 (e.g., a structure module in Alphafold2). As an example shown in FIG. 10, the encoder 1052 can use row-wise gated self-attention3, triangle update, and triangle self-attention and the decoder 1054 uses Invariant Point Attention to learn amino acid interactions and geometry representations. In this example, the encoder 1052 includes 48 blocks and the decoder 1054 includes 8 blocks.

Similar to xTrimoABFold 100, xTrimoABFold++ 1000 can be trained end to end using the various loss functions described above. For example, the loss function of xTrimoABFold++ 1000 can include the CDR focal loss and the RMSD loss as discussed w.r.t. Equations (9) and (10) in addition to or as an alternative to some of the losses used in existing protein structure prediction models.

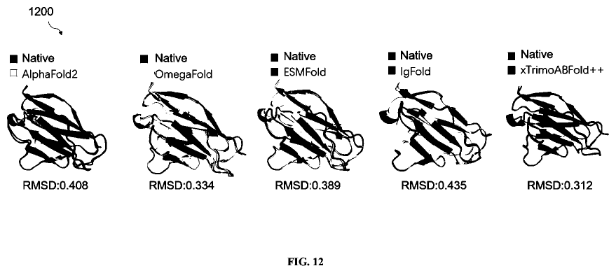

FIG. 11 includes Table 5 illustrating accuracy performances of different example protein structure prediction models including xTrimoABFold++ 1000 on antibody structure prediction, in accordance with embodiments of this specification. As shown, xTrimoABFold++ outperforms all baselines on antibody structure prediction, especially for CDR-H3 on an antibody dataset consisting of 68 antibody complexes.

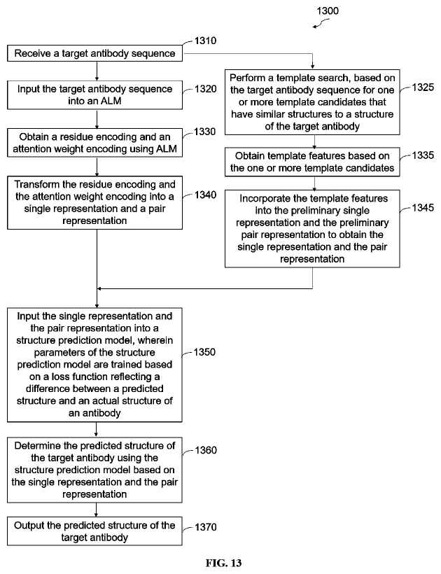

FIG. 12 is a plot 1200 illustrating examples of protein structures predicted by the xTrimoABFold++ and other baselines, in accordance with embodiments of this specification. The plot 1200 shows an example of a target protein, PDB 7WVM_B, the light chain of cemiplimab for PD-1. As shown, xTrimoABFold++ outperforms other baselines on in terms of RMSD.

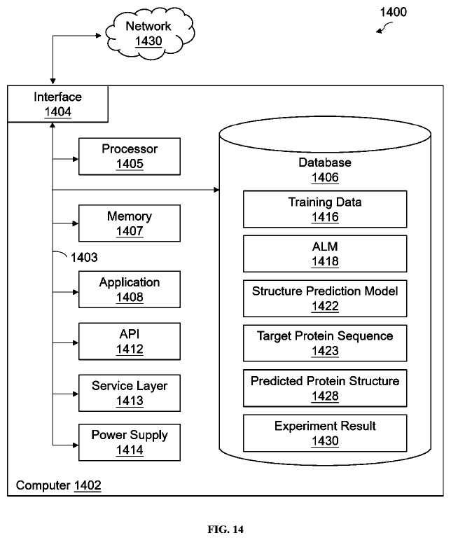

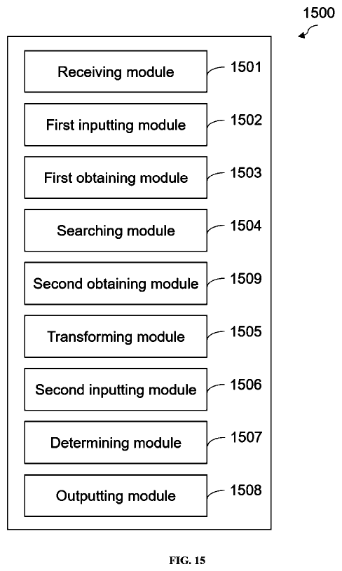

FIG. 13 is a flowchart of an example process 1300 for protein structure prediction, in accordance with embodiments of this specification. The process 1300 can be an example of an MSA-free protein structure prediction algorithm performed by a data processing apparatus, such as a computer-implemented system 100 in FIG. 1 or computer-implemented system 1000 in FIG. 10. In some embodiments, a data processing apparatus can be a system of one or more computers, located in one or more locations, and programmed appropriately in accordance with this specification. For example, a computer-implemented system 1400 of FIG. 14, appropriately programmed, can perform the example process 1300.

In some embodiments, the example process 1300 shown in FIG. 13 can be modified or reconfigured to include additional, fewer, or different operations, which can be performed in the order shown or in a different order. In some instances, one or more of the operations can be repeated or iterated, for example, until a terminating condition is reached. In some implementations, one or more of the individual operations shown in FIG. 13 can be executed as multiple separate operations, or one or more subsets of the operations shown in FIG. 13 can be combined and executed as a single operation.

Although FIG. 13 is described referring to antibodies and antibody sequences (e.g., a target antibody sequence), the example process 1300 can be applied more generally for protein structure prediction, for example, based on a target protein sequence.

At 1310, a target antibody sequence that includes a sequence of amino acids (or amino acid residues) is input, configured, identified, obtained, or otherwise received by the data processing apparatus. The target antibody sequence can represent an antibody that is specified by the sequence of amino acids. The example process 1300 can be used to predict a structure of the antibody that is specified by the sequence of amino acids. The target antibody sequence can be the example amino acid sequence or residue sequence 110 or 1010.

In some embodiments, receiving the target antibody sequence includes receiving data representing the target antibody sequence. For example, data representing the target antibody sequence can include embeddings that represent the amino acids in the target antibody sequence. An “embedding” can be an ordered collection of numerical values, e.g., a vector, matrix, tensor of numerical values. Accordingly, the target antibody sequence can be represented as a vector, matrix, tensor, or another form or data structure. In some embodiments, the target antibody sequence includes additional data such as embedding data (e.g., one-hot encoding data) associated with the target antibody sequence. As an example, different amino acids can be represented by different letters, e.g., A to Z. For each amino acid, corresponding embedding data can be word2vec vectors or another type of embedding code. Accordingly, a antibody composed of amino acids can be represented by the respective letter representations and/or embedding data representations of the amino acids. In some embodiments, amino acids and the antibody can be represented in another manner or data structure for computer processing.

At 1320, the target antibody sequence is input into an ALM. The ALM can be a protein language model trained from antibody sequences. The ALM can be the example ALM 130, 230, or 1030.

For example, the ALM can be trained using an antibody database that comprises antibody sequences or consisting only antibody sequences. In some embodiments, the ALM can be pre-trained, for example, independently or separately from the overall model configured for protein structure prediction. In some embodiments, the ALM can be trained or fine-tuned as part of an overall model configured for protein structure prediction (e.g., the xTrimoABFold 100 or xTrimoABFold++ 1000) using a loss function (e.g., one or more of the loss function in Equation (5), (8), (9) or (10)) of the overall model. In the latter case, parameters of the first machine learning model and second machine learning model can be trained or updated based on a gradient of the loss function of the overall model configured for protein structure prediction.

In some embodiments, the ALM can be a neural network such as a self-attention model that includes a plurality of self-attention neural network layers (also referred to as self-attention layers). Various types of a self-attention models or architectures can be used as a basis to train the ALM. In some embodiments, the ALM is pre-trained using an antibody database according to a Bidirectional Encoder Representations from Transformers (BERT) architecture, such as e.g., an AntiBERTy architecture.

At 1330, a residue encoding and an attention weight encoding are obtained using the ALM without performing multiple sequence alignment (MSA). The residue encoding is used to generate a single representation to be input into a structure prediction model (e.g., the structure prediction model 150 or 1050). The attention weight encoding is used to generate a pair representation to be input into the structure prediction model (e.g., the structure prediction model 150 or 1050).

The residue encoding can be a residue-level data representation that includes a respective first embedding corresponding to each amino acid in the target antibody sequence. The respective first embedding is output by the ALM by using the target antibody sequence as the input to the ALM, for example, according to the example techniques described w.r.t. FIGS. 1, 2 and 10. For example, the residue encoding can be the example residue encoding 125, the output 250, or the last hidden states 1025. The residue encoding can be represented by a vector, matrix, tensor of numerical values, or another data structure. Unlike conventional protein structure prediction approaches that generate single representations based on MSA embeddings, the residue encoding is output by the ALM without performing MSA, and thus improve computational efficiency of the process 1300.

The attention weight encoding can be a pairwise data representation that includes a respective second embedding corresponding to a pair of amino acids in the target antibody sequence. If the number of residues in the sequence is N, the number of pairs and the size of the attention weight encoding is N*N. The respective second embedding is calculated from attention weights of the self-attention layers of the ALM. For example, the attention weight encoding can include the example attention weight encoding 135 or attention weights 1035, for example, according to the example techniques described w.r.t. FIGS. 1, 3 and 10.

The attention weight encoding can be represented by a vector, matrix, tensor of numerical values, or another data structure. Unlike conventional protein structure prediction approaches that generate pair representations based on MSA embeddings, the attention weight encoding is generated based on the attention weights of the ALM, without using MSA embeddings, and thus improve computational efficiency of the process 1300.

In some embodiments, if the ALM comprises L self-attention layers, each of the L self-attention layers comprises H attention heads, the attention weight encoding can include an second embedding (e.g., qᵢⱼ in Equation (2-4)) corresponding to a pair of an amino acid i and an amino acid j in the target antibody sequence. Obtaining, using the ALM without performing MSA, the second embedding qᵢⱼ comprises obtaining attention weights of the H attention heads of the each of the L self-attention layers when the amino acid i is used as a query and the amino acid j is used as a key in the ALM; and concatenating the attention weights to obtain the second embedding qᵢⱼ, for example, according to Equation (2-4). In some embodiments, the embedding qᵢⱼ can include attention weights of the H attention heads of the L layers concatenated, collected, or assembled in another manner. The attention weights can be computed based on a query-key product

$$( e . g . , {{Q}_{i}^{h , l} ( {K}_{j}^{h , l} )}^{T} )$$

when the amino acid i is used as a query and the amino acid j is used as a key in the ALM. The attention weights can be A^{h,l} that is calculated, for example, according to a softmax operation as shown in Equation (2-3), another normalization operation of B^{h,l}, or another variant of B^{h,l} or B^{h,l} itself.

At 1340, the residue encoding and the attention weight encoding are transformed into a single representation and a pair representation. The single representation can include data representing features corresponding to a single residue in the sequence of amino acids of the target antibody sequence. The pair representation can include data representing features corresponding to a pair of residues in the sequence of amino acids of the target antibody sequence. The single representation and the pair representation can be represented in the form of vectors, matrices, tensors, or other data structures. The single representation and the pair representation can be an initial single representation (e.g., initial single representation 175 or 1175) and an initial pair representation (e.g., initial pair representation 185 or 1185) to be input into a structure prediction model. In some embodiments, transforming, by the data processing apparatus, the residue encoding and the attention weight encoding into a single representation and a pair representation comprises: transforming the residue encoding into the single representation by a first machine learning model such as a first linear neural network layer (e.g., FCNN 1045); and transforming the attention weight encoding into the pair representation by a first machine learning model such as a second linear neural network layer (e.g., FCNN 1055). The first machine learning model and second machine learning model can be trained individually or as part of an overall model configured for protein structure prediction (e.g., the xTrimoABFold 100 or xTrimoABFold++ 1000) using a loss function (e.g., one or more of the loss function in Equation (5), (8), (9) or (10)) of the overall model. In the latter case, parameters of the first machine learning model and second machine learning model can be trained, for example, by updating the parameters based on a gradient of the loss function of the overall model configured for protein structure prediction.

In some embodiments, the example process 1300 further includes a template search to identify one or more template candidates that have similar structures to the target antibody. The one or more template candidates can be used to initialize the single representation and the pair representation before the single representation and the pair representation are input into the structure prediction model. In some embodiments, steps 1325, 1335, and 1345 related to the template search can be performed.

At 1325, a template search is performed, based on the target antibody sequence without multiple sequence alignment (MSA), for one or more template candidates that have similar structures to the target antibody. The template search can using the example cross-modal template searching algorithm as described w.r.t. FIG. 1, or another template searching algorithm. For example, performing the template search for one or more template candidates comprises performing a sequential modal search in a first structure database for first structure templates, wherein sequences of antibodies corresponding to the first structure templates are similar to the target antibody sequence; and performing a structural modal search in a second structure database for second structure templates, wherein structures of the second structure templates are similar to a coarse-grained structure of the target antibody sequence. The one or more template candidates comprise the first structure templates and/or the second structure templates. The first structure database and the second structure database can be the same of different.

At 1335, template features (e.g., template features 165) are obtained based on the one or more template candidates. The template features can be obtained, for example, by extracting matching features from the one or more template candidates to be added or otherwise incorporated into corresponding features in the single representation and the pair representation.

At 1345, the template features are incorporated into the single representation and the pair representation generated at step 1340. For example, the single representation and the pair representation generated at step 1340 can be regarded as generated an preliminary single representation and an preliminary pair representation, and the template features are added into the preliminary single representation and the preliminary pair representation.

In some embodiments, the process 1300 does not include any template search (e.g., any of the steps 1325, 1335, and 1345). In this case, the single representation and the pair representation do not incorporate template features before the single representation and the pair representation are input into the structure prediction model.

At 1350, the single representation and the pair representation are input into a structure prediction model (e.g., the structure prediction model 150 or 1050). Parameters of the structure prediction model are trained or otherwise obtained based on a loss function reflecting a difference between a predicted structure and an actual structure of an antibody. As an example, the parameters of the structure prediction model are trained by solving an optimization problem to minimize the loss function, for example, by updating the parameters based on a gradient of the loss function. The loss function can be one or more of the loss function in Equation (5), (8), (9) or (10), or can include additional or different losses. However, the loss function does not comprise a loss due to MSA. As an example, the loss function comprises a framed aligned point error (FAPE) loss and a torsion angle loss, and a loss focusing on a complementarity determining region (CDR). The loss represents a difference between the predicted structure and an actual structure of the target antibody. As another example, the loss function comprises a differential root-mean-squared-deviation (RMSD) in addition to or in place of a framed aligned point error (FAPE) loss between the predicted structure and an actual structure of the target antibody sequence.

At 1350, the predicted structure of the target antibody is determined using the structure prediction model based on the single representation and the pair representation. For example, after the overall model configured for protein structure prediction (e.g., the xTrimoABFold 100 or xTrimoABFold++ 1000) that includes the ALM and the structure prediction model is trained, the predicted structure of the target antibody is determined using the structure prediction model in the interference phase. In some embodiments, the predicted structure of the target antibody sequence is determined using the structure prediction model in an iterative manner until a convergence or another terminating condition (e.g., the number of iterations) is met.

At 1360, the predicted structure of the target antibody is output. The predicted structure of the target antibody can be defined by values of a plurality of structure parameters such as atoms positions and angles to represent a 3D structure of the target antibody specified by the target antibody sequence. In some embodiments, experiments, testing, and further processing such as drug discovery and design, can be performed based on the predicted structure of the target antibody.

FIG. 14 is a block diagram illustrating an example of a computer-implemented system 1400 used to provide computational functionalities associated with described algorithms, methods, functions, processes, flows, and procedures, according to an embodiment of the present disclosure. For example, System 1400 can be an example of data processing apparatus configured to perform protein structure prediction, in accordance with embodiments of this specification. In the illustrated embodiment, System 1400 includes a Computer 1402 and a Network 1430.

The illustrated Computer 1402 is intended to encompass any computing device such as a server, desktop computer, laptop/notebook computer, wireless data port, smart phone, personal data assistant (PDA), tablet computer, one or more processors within these devices, another computing device, or a combination of computing devices, including physical or virtual instances of the computing device, or a combination of physical or virtual instances of the computing device. Additionally, the Computer 1402 can include an input device, such as a keypad, keyboard, touch screen, another input device, or a combination of input devices that can accept user information, and an output device that conveys information associated with the operation of the Computer 1402, including digital data, visual, audio, another type of information, or a combination of types of information, on a graphical-type user interface (UI) (or GUI) or other UI.

The Computer 1402 can serve in a role in a distributed computing system as a client, network component, a server, a database or another persistency, another role, or a combination of roles for performing the subject matter described in the present disclosure. The illustrated Computer 1402 is communicably coupled with a Network 1430. In some embodiments, one or more components of the Computer 1402 can be configured to operate within an environment, including cloud-computing-based, local, global, another environment, or a combination of environments.

At a high level, the Computer 1402 is an electronic computing device operable to receive, transmit, process, store, or manage data and information associated with the described subject matter. According to some embodiments, the Computer 1402 can also include or be communicably coupled with a server, including an application server, e-mail server, web server, caching server, streaming data server, another server, or a combination of servers.

The Computer 1402 can receive requests over Network 1430 (for example, from a client software application executing on another Computer 1402) and respond to the received requests by processing the received requests using a software application or a combination of software applications. In addition, requests can also be sent to the Computer 1402 from internal users (for example, from a command console or by another internal access method), external or third-parties, or other entities, individuals, systems, or computers.

Each of the components of the Computer 1402 can communicate using a System Bus 1403. In some embodiments, any or all of the components of the Computer 1402, including hardware, software, or a combination of hardware and software, can interface over the System Bus 1403 using an application programming interface (API) 1412, a Service Layer 1413, or a combination of the API 1412 and Service Layer 1413. The API 1412 can include specifications for routines, data structures, and object classes. The API 1412 can be either computer-language independent or dependent and refer to a complete interface, a single function, or even a set of APIs. The Service Layer 1413 provides software services to the Computer 1402 or other components (whether illustrated or not) that are communicably coupled to the Computer 1402. The functionality of the Computer 1402 can be accessible for all service consumers using the Service Layer 1413. Software services, such as those provided by the Service Layer 1413, provide reusable, defined functionalities through a defined interface. For example, the interface can be software written in JAVA, C++, another computing language, or a combination of computing languages providing data in extensible markup language (XML) format, another format, or a combination of formats. While illustrated as an integrated component of the Computer 1402, alternative embodiments can illustrate the API 1412 or the Service Layer 1413 as stand-alone components in relation to other components of the Computer 1402 or other components (whether illustrated or not) that are communicably coupled to the Computer 1402. Moreover, any or all parts of the API 1412 or the Service Layer 1413 can be implemented as a child or a sub-module of another software module, enterprise application, or hardware module without departing from the scope of the present disclosure.

The Computer 1402 includes an Interface 1404. Although illustrated as a single Interface 1404, two or more Interfaces 1404 can be used according to particular needs, desires, or particular embodiments of the Computer 1402. The Interface 1404 is used by the Computer 1402 for communicating with another computing system (whether illustrated or not) that is communicatively linked to the Network 1430 in a distributed environment. Generally, the Interface 1404 is operable to communicate with the Network 1430 and includes logic encoded in software, hardware, or a combination of software and hardware. More specifically, the Interface 1404 can include software supporting one or more communication protocols associated with communications such that the Network 1430 or hardware of Interface 1404 is operable to communicate physical signals within and outside of the illustrated Computer 1402.

The Computer 1402 includes a Processor 1405. Although illustrated as a single Processor 1405, two or more Processors 1405 can be used according to particular needs, desires, or particular embodiments of the Computer 1402. Generally, the Processor 1405 executes instructions and manipulates data to perform the operations of the Computer 1402 and any algorithms, methods, functions, processes, flows, and procedures as described in the present disclosure.

The Computer 1402 also includes a Database 1406 that can hold data for the Computer 1402, another component communicatively linked to the Network 1430 (whether illustrated or not), or a combination of the Computer 1402 and another component. For example, Database 1406 can be an in-memory, conventional, or another type of database storing data consistent with the present disclosure. In some embodiments, Database 1406 can be a combination of two or more different database types (for example, a hybrid in-memory and conventional database) according to particular needs, desires, or particular embodiments of the Computer 1402 and the described functionality. Although illustrated as a single Database 1406, two or more databases of similar or differing types can be used according to particular needs, desires, or particular embodiments of the Computer 1402 and the described functionality. While Database 1406 is illustrated as an integral component of the Computer 1402, in alternative embodiments, Database 1406 can be external to the Computer 1402.

As an example, Database 1406 can store data referenced with embodiments of this specification. For example, Database 1406 can store one or more of a database (e.g., antibody structure dataset 105 and the template database 115), training data 1416 for training the ALM and/or an overall model configured for protein structure prediction (e.g., the xTrimoABFold 100 or xTrimoABFold++ 1000), a pre-trained ALM 1418 (e.g., the ALM 130, 230, or 1030), a structure prediction model 1422 (e.g., the structure prediction model 150 or 150), or another component or sub-model (e.g., FCNN 1045 or 1055) of the overall model configured for protein structure prediction, a target proteins 1423 (e.g., the target protein sequence 110, 210, or 1010), a predicted protein structure 1428, or other testing/experiment results 1432.

The Computer 1402 also includes a Memory 1407 that can hold data for the Computer 1402, another component or components communicatively linked to the Network 1430 (whether illustrated or not), or a combination of the Computer 1402 and another component. Memory 1407 can store any data consistent with the present disclosure. In some embodiments, Memory 1407 can be a combination of two or more different types of memory (for example, a combination of semiconductor and magnetic storage) according to particular needs, desires, or particular embodiments of the Computer 1402 and the described functionality. Although illustrated as a single Memory 1407, two or more Memories 1407 or similar or differing types can be used according to particular needs, desires, or particular embodiments of the Computer 1402 and the described functionality. While Memory 1407 is illustrated as an integral component of the Computer 1402, in alternative embodiments, Memory 1407 can be external to the Computer 1402.

The Application 1408 is an algorithmic software engine providing functionality according to particular needs, desires, or particular embodiments of the Computer 1402, particularly with respect to functionality described in the present disclosure. For example, Application 1408 can serve as one or more components, modules, or applications. Further, although illustrated as a single Application 1408, the Application 1408 can be implemented as multiple Applications 1408 on the Computer 1402. In addition, although illustrated as integral to the Computer 1402, in alternative embodiments, the Application 1408 can be external to the Computer 1402.

The Computer 1402 can also include a Power Supply 1414. The Power Supply 1414 can include a rechargeable or non-rechargeable battery that can be configured to be either user- or non-user-replaceable. In some embodiments, the Power Supply 1414 can include power-conversion or management circuits (including recharging, standby, or another power management functionality). In some embodiments, the Power Supply 1414 can include a power plug to allow the Computer 1402 to be plugged into a wall socket or another power source to, for example, power the Computer 1402 or recharge a rechargeable battery.

There can be any number of Computers 1402 associated with, or external to, a computer system containing Computer 1402, each Computer 1402 communicating over Network 1430. Further, the term “client,” “user,” or other appropriate terminology can be used interchangeably, as appropriate, without departing from the scope of the present disclosure. Moreover, the present disclosure contemplates that many users can use one Computer 1402, or that one user can use multiple computers 1402.

FIG. 15 is a diagram of an example of modules of an apparatus 1500 in accordance with embodiments of this specification. The apparatus 1500 can be an example embodiment of a data processing apparatus for protein structure prediction, in accordance with embodiments of this specification. The apparatus 1500 can correspond to the embodiments described above, and the apparatus 1500 includes the following: a receiving module 1501 that receives a target antibody sequence of a target antibody that includes a sequence of amino acids, a first input module 1502 that inputs the target antibody sequence into an antibody language model (ALM), wherein the ALM is a protein language model trained from antibody sequences, and the ALM comprises a plurality of self-attention layers, an obtaining module 1503 that obtains a residue encoding and an attention weight encoding using the ALM without performing multiple sequence alignment (MSA), a transforming module 1505 that transforms the residue encoding and the attention weight encoding into a single representation and a pair representation; a second input module 1506 that inputs the single representation and the pair representation into a structure prediction model, wherein parameters of the structure prediction model are trained based on a loss function reflecting a difference between a predicted structure and an actual structure of an antibody, a determining module 1507 that determines the predicted structure of the target antibody using the structure prediction model based on the single representation and the pair representation, and an outputting module 1508 that outputs the predicted structure of the target antibody.

In some embodiments, the apparatus 1500 further includes the following: a searching module 1504 that performs a template search, based on the target antibody sequence without multiple sequence alignment (MSA), for one or more template candidates that have similar structures to a structure of the target antibody before inputting the single representation and the pair representation into the structure prediction model; and a second obtaining module 1509 that obtains template features based on the one or more template candidates; and wherein transforming the residue encoding and the attention weight encoding into the single representation and the pair representation comprises: transforming the residue encoding and the attention weight encoding into a preliminary single representation and an preliminary pair representation; incorporating the template features into the preliminary single representation and the preliminary pair representation to obtain the single representation and the pair representation.

In some embodiments, wherein performing the template search for one or more template candidates comprises: performing a sequential modal search in a first structure database for first structure templates, wherein sequences of antibodies corresponding to the first structure templates are similar to the target antibody sequence; and performing a structural modal search in a second structure database for second structure templates, wherein structures of the second structure templates are similar to a coarse-grained structure of the target antibody sequence, and wherein the one or more template candidates comprise one or more of the first structure templates or the second structure templates, and wherein the coarse-grained structure is a default structure or a structure predicted from another structure prediction algorithm or another structure prediction model.

In some embodiments, wherein the ALM is pre-trained using an antibody database according to a Bidirectional Encoder Representations from Transformers (BERT) architecture, and the antibody database consists the antibody sequences.

In some embodiments, wherein the ALM comprises L self-attention layers, each of the L self-attention layers comprises H attention heads, and wherein a second embedding qij corresponding to a pair of an amino acid i and an amino acid j in the target antibody sequence, and wherein obtaining, by the data processing apparatus using the ALM without performing MSA, the attention weight encoding comprises: obtaining attention weights of the H attention heads of the each of the L self-attention layers when the amino acid i is used as a query and the amino acid j is used as a key; and concatenating the attention weights to obtain the second embedding qij.

In some embodiments, wherein transforming the residue encoding and the attention weight encoding into a single representation and a pair representation comprises: transforming the residue encoding into the single representation by a first linear neural network layer; and transforming the attention weight encoding into the pair representation by a second linear neural network layer; wherein parameters of the first linear neural network layer and the second linear neural network layer are updated based on a gradient of the loss function.

In some embodiments, wherein the loss function does not comprise a loss due to MSA.

In some embodiments, wherein the loss function comprises a framed aligned point error (FAPE) loss and a torsion angle loss, and a loss focusing on a complementarity determining region (CDR).

In some embodiments, wherein the loss function comprises a differential root-mean-squared-deviation (RMSD) in addition to a framed aligned point error (FAPE) loss.

In some embodiments, wherein the single representation and the pair representation do not incorporate template features before the single representation and the pair representation are input into the structure prediction model.

Described embodiments of the subject matter can include one or more features, alone or in combination. For example, in a first embodiment, a computer-implemented method for antibody structure prediction includes one or more of the following: a target antibody sequence of a target antibody that includes a sequence of amino acids is received. The target antibody sequence is input into an antibody language model (ALM), wherein the ALM is a protein language model trained from antibody sequences, and the ALM comprises a plurality of self-attention layers. A residue encoding and an attention weight encoding are obtained using the ALM without performing multiple sequence alignment (MSA), wherein the residue encoding comprises a respective first embedding corresponding to each of the amino acids in the target antibody sequence output by the ALM; and the attention weight encoding comprises a respective second embedding corresponding to a pair of amino acids in the target antibody sequence calculated from attention weights of the self-attention layers of the ALM. The residue encoding and the attention weight encoding are transformed into a single representation and a pair representation. The single representation and the pair representation are input into a structure prediction model, wherein parameters of the structure prediction model are trained based on a loss function reflecting a difference between a predicted structure and an actual structure of an antibody. The predicted structure of the target antibody is determined using the structure prediction model based on the single representation and the pair representation. The predicted structure of the target antibody is output.

The foregoing and other described embodiments can each, optionally, include one or more of the following features:

A first feature, combinable with any of the following features, specifies that the ALM is pre-trained using an antibody database according to a Bidirectional Encoder Representations from Transformers (BERT) architecture, and the antibody database consists the antibody sequences.

A second feature, combinable with any of the following features, specifies that the ALM comprises L self-attention layers, each of the L self-attention layers comprises H attention heads, and wherein a second embedding qij corresponding to a pair of an amino acid i and an amino acid j in the target antibody sequence, and wherein obtaining, using the ALM without performing MSA, the attention weight encoding comprises: obtaining attention weights of the H attention heads of the each of the L self-attention layers when the amino acid i is used as a query and the amino acid j is used as a key; and concatenating the attention weights to obtain the second embedding qij.

A third feature, combinable with any of the following features, specifies that transforming the residue encoding and the attention weight encoding into a single representation and a pair representation comprises: transforming the residue encoding into the single representation by a first linear neural network layer; and transforming the attention weight encoding into the pair representation by a second linear neural network layer; wherein parameters of the first linear neural network layer and the second linear neural network layer are updated based on a gradient of the loss function.

A fourth feature, combinable with any of the following features, specifies that the loss function does not comprise a loss due to MSA.

A fifth feature, combinable with any of the following features, specifies that wherein the loss function comprises a framed aligned point error (FAPE) loss and a torsion angle loss, and a loss focusing on a complementarity determining region (CDR).

A sixth feature, combinable with any of the following features, specifies that the loss function comprises a differential root-mean-squared-deviation (RMSD) in addition to a framed aligned point error (FAPE) loss.

A seventh feature, combinable with any of the following features, specifies that the single representation and the pair representation do not incorporate template features before the single representation and the pair representation are input into the structure prediction model.

An eighth feature, combinable with any of the following features, specifies that wherein, before inputting the single representation and the pair representation into the structure prediction model, the computer-implemented method further comprises: performing a template search, based on the target antibody sequence without multiple sequence alignment (MSA), for one or more template candidates that have similar structures to a structure of the target antibody; and obtaining template features based on the one or more template candidates; and wherein transforming the residue encoding and the attention weight encoding into the single representation and the pair representation comprises: transforming the residue encoding and the attention weight encoding into a preliminary single representation and an preliminary pair representation; incorporating the template features into the preliminary single representation and the preliminary pair representation to obtain the single representation and the pair representation.

A nineth feature, combinable with any of the following features, specifies that performing the template search for one or more template candidates comprises: performing a sequential modal search in a first structure database for first structure templates, wherein sequences of antibodies corresponding to the first structure templates are similar to the target antibody sequence; and performing a structural modal search in a second structure database for second structure templates, wherein structures of the second structure templates are similar to a coarse-grained structure of the target antibody sequence, and wherein the one or more template candidates comprise one or more of the first structure templates or the second structure templates, and wherein the coarse-grained structure is a default structure or a structure predicted from another structure prediction algorithm or another structure prediction model.

In a second embodiment, a system, including: one or more processors; and one or more computer-readable memories coupled to the one or more processors and having instructions stored thereon which are executable by the one or more processors to perform the method of any of the first embodiment and its optional combination of the one or more of features described above.

In a third embodiment, an apparatus for identifying a target protein corresponding to an object protein. The apparatus includes one or more modules (e.g., the modules as described w.r.t. FIG. 15) for performing the method of any of the first embodiment and its optional combination of the one or more of features described above.

The system, apparatus, module, or unit illustrated in the previous embodiments can be implemented by using a computer chip or an entity, or can be implemented by using a product having a certain function. A typical embodiment device is a computer (and the computer can be a personal computer), a laptop computer, a cellular phone, a camera phone, a smartphone, a personal digital assistant, a media player, a navigation device, an email receiving and sending device, a game console, a tablet computer, a wearable device, or any combination of these devices.

For an embodiment process of functions and roles of each module in the apparatus, references can be made to an embodiment process of corresponding steps in the previous method. Details are omitted here for simplicity.

Because an apparatus embodiment basically corresponds to a method embodiment, for related parts, references can be made to related descriptions in the method embodiment. The previously described apparatus embodiment is merely an example. The modules described as separate parts may or may not be physically separate, and parts displayed as modules may or may not be physical modules, may be located in one position, or may be distributed on a number of network modules. Some or all of the modules can be selected based on actual demands to achieve the objectives of the solutions of the specification. A person of ordinary skill in the art can understand and implement the embodiments of the present application without creative efforts.

Referring again to FIG. 15, it can be interpreted as illustrating internal functional modules and a structure of a computing implementation apparatus. The computing implementation apparatus can be an example of a computing system configured to identify a target protein corresponding to an object protein. An execution body in essence can be an electronic device, and the electronic device includes the following: one or more processors; and one or more computer-readable memories configured to store an executable instruction of the one or more processors. In some embodiments, the one or more computer-readable memories are coupled to the one or more processors and have programming instructions stored thereon that are executable by the one or more processors to perform algorithms, methods, functions, processes, flows, and procedures, as described in this specification. This specification also provides one or more non-transitory computer-readable storage media coupled to one or more processors and having instructions stored thereon which, when executed by the one or more processors, cause the one or more processors to perform operations in accordance with embodiments of the methods provided herein.

This specification further provides a system for implementing the methods provided herein. The system includes one or more processors, and a computer-readable storage medium coupled to the one or more processors having instructions stored thereon which, when executed by the one or more processors, cause the one or more processors to perform operations in accordance with embodiments of the methods provided herein.

Embodiments of the subject matter and the actions and operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, e.g., one or more modules of computer program instructions, encoded on a computer program carrier, for execution by, or to control the operation of, data processing apparatus. For example, a computer program carrier can include one or more computer-readable storage media that have instructions encoded or stored thereon. The carrier may be a tangible non-transitory computer-readable medium, such as a magnetic, magneto optical, or optical disk, a solid state drive, a random access memory (RAM), a read-only memory (ROM), or other types of media.

Alternatively, or in addition, the carrier may be an artificially generated propagated signal, e.g., a machine-generated electrical, optical, or electromagnetic signal that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

A computer program, which may also be referred to or described as a program, software, a software application, an app, a module, a software module, an engine, a script, or code, can be written in any form of programming language, including compiled or interpreted languages, or declarative or procedural languages; and it can be deployed in any form, including as a stand-alone program or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub programs, or portions of code.

Processors for execution of a computer program include, by way of example, both general- and special-purpose microprocessors, and any one or more processors of any kind of digital computer. Generally, a processor will receive the instructions of the computer program for execution as well as data from a non-transitory computer-readable medium coupled to the processor.

The term “data processing apparatus” encompasses all kinds of apparatuses, devices, and machines for processing data, including by way of example a programmable processor, a computer, or multiple processors or computers. Data processing apparatus can include special-purpose logic circuitry, e.g., an FPGA (field programmable gate array), an ASIC (application specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

The processes and logic flows described in this specification can be performed by the data processing apparatus as a software, hardware, firmware, or hybrid implementation. For example, the processes and logic flows described in this specification can be performed by one or more computers or processors executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random access memory or both. Elements of a computer can include a central processing unit for executing instructions and one or more memory devices for storing instructions and data. The central processing unit and the memory can be supplemented by, or incorporated in, special-purpose logic circuitry.

Generally, a computer will also include, or be operatively coupled to receive data from or transfer data to one or more storage devices. The storage devices can be, for example, magnetic, magneto optical, or optical disks, solid state drives, or any other type of non-transitory, computer-readable media. However, a computer need not have such devices. Thus, a computer may be coupled to one or more storage devices, such as, one or more memories, that are local and/or remote. For example, a computer can include one or more local memories that are integral components of the computer, or the computer can be coupled to one or more remote memories that are in a cloud network. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Components can be “coupled to” each other by being commutatively such as electrically or optically connected to one another, either directly or via one or more intermediate components. Components can also be “coupled to” each other if one of the components is integrated into the other. For example, a storage component that is integrated into a processor (e.g., an L2 cache component) is “coupled to” the processor.

To provide for interaction with a user, embodiments of the subject matter described in this specification can be implemented on, or configured to communicate with, a computer having a display device, e.g., an LCD (liquid crystal display) monitor, for displaying information to the user, and an input device by which the user can provide input to the computer, e.g., a keyboard and a pointing device, e.g., a mouse, a trackball, or touchpad. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

This specification uses the term “configured to” in connection with systems, apparatus, and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions. For special-purpose logic circuitry to be configured to perform particular operations or actions means that the circuitry has electronic logic that performs the operations or actions.

While this specification contains many specific embodiment details, these should not be construed as limitations on the scope of what is being claimed, which can be computed by the claims themselves, but rather as descriptions of features that may be specific to particular embodiments. Certain features that are described in this specification in the context of separate embodiments can also be realized in combination in a single embodiment. Conversely, various features that are described in the context of a single embodiments can also be realized in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A computer-implemented method for antibody structure prediction, wherein a predicted structure of a given antibody is defined by values of a plurality of structure parameters, the method comprising:
receiving, by a data processing apparatus, a target antibody sequence of a target antibody that includes a sequence of amino acids;
inputting, by the data processing apparatus, the target antibody sequence into an antibody language model (ALM), wherein the ALM is a protein language model trained from antibody sequences, and the ALM comprises a plurality of self-attention layers;
obtaining, by the data processing apparatus using the ALM without performing multiple sequence alignment (MSA), a residue encoding and an attention weight encoding, wherein:
the residue encoding comprises a respective first embedding corresponding to each of the amino acids in the target antibody sequence output by the ALM; and
the attention weight encoding comprises a respective second embedding corresponding to a pair of amino acids in the target antibody sequence calculated from attention weights of the self-attention layers of the ALM;
transforming, by the data processing apparatus, the residue encoding and the attention weight encoding into a single representation and a pair representation;
inputting, by the data processing apparatus, the single representation and the pair representation into a structure prediction model, wherein parameters of the structure prediction model are trained based on a loss function reflecting a difference between a predicted structure and an actual structure of an antibody;
determining, by the data processing apparatus, the predicted structure of the target antibody using the structure prediction model based on the single representation and the pair representation; and
outputting, by the data processing apparatus, the predicted structure of the target antibody.

2. The computer-implemented method of claim 1, wherein the ALM is pre-trained using an antibody database according to a Bidirectional Encoder Representations from Transformers (BERT) architecture, and the antibody database consists the antibody sequences.

3. The computer-implemented method of claim 1, wherein the ALM comprises L self-attention layers, each of the L self-attention layers comprises H attention heads, and
wherein a second embedding qᵢⱼ corresponding to a pair of an amino acid i and an amino acid j in the target antibody sequence, and
wherein obtaining, by the data processing apparatus using the ALM without performing MSA, the attention weight encoding comprises:
obtaining attention weights of the H attention heads of the each of the L self-attention layers when the amino acid i is used as a query and the amino acid j is used as a key; and
concatenating the attention weights to obtain the second embedding qᵢⱼ.

4. The computer-implemented method of claim 1, wherein transforming, by the data processing apparatus, the residue encoding and the attention weight encoding into a single representation and a pair representation comprises:
transforming the residue encoding into the single representation by a first linear neural network layer; and
transforming the attention weight encoding into the pair representation by a second linear neural network layer;
wherein parameters of the first linear neural network layer and the second linear neural network layer are updated based on a gradient of the loss function.

5. The computer-implemented method of claim 1, wherein the loss function does not comprise a loss due to MSA.

6. The computer-implemented method of claim 1, wherein the loss function comprises a framed aligned point error (FAPE) loss and a torsion angle loss, and a loss focusing on a complementarity determining region (CDR).

7. The computer-implemented method of claim 1, wherein the loss function comprises a differential root-mean-squared-deviation (RMSD) in addition to a framed aligned point error (FAPE) loss.

8. The computer-implemented method of claim 1, wherein the single representation and the pair representation do not incorporate template features before the single representation and the pair representation are input into the structure prediction model.

9. The computer-implemented method of claim 1, wherein, before inputting, by the data processing apparatus, the single representation and the pair representation into the structure prediction model, the computer-implemented method further comprises:
performing, by the data processing apparatus, a template search, based on the target antibody sequence without multiple sequence alignment (MSA), for one or more template candidates that have similar structures to a structure of the target antibody; and
obtaining, by the data processing apparatus, template features based on the one or more template candidates; and
wherein transforming, by the data processing apparatus, the residue encoding and the attention weight encoding into the single representation and the pair representation comprises:
transforming, by the data processing apparatus, the residue encoding and the attention weight encoding into a preliminary single representation and an preliminary pair representation; and
incorporating, by the data processing apparatus, the template features into the preliminary single representation and the preliminary pair representation to obtain the single representation and the pair representation.

10. The computer-implemented method of claim 9, wherein performing, by the data processing apparatus, the template search for one or more template candidates comprises:
performing, by the data processing apparatus, a sequential modal search in a first structure database for first structure templates, wherein sequences of antibodies corresponding to the first structure templates are similar to the target antibody sequence; and
performing, by the data processing apparatus, a structural modal search in a second structure database for second structure templates, wherein structures of the second structure templates are similar to a coarse-grained structure of the target antibody sequence, and
wherein the one or more template candidates comprise one or more of the first structure templates or the second structure templates, and
wherein the coarse-grained structure is a default structure or a structure predicted from another structure prediction algorithm or another structure prediction model.

11. A system for performing a software-implemented application for antibody structure prediction, wherein a predicted structure of a given antibody is defined by values of a plurality of structure parameters, the system comprising:
one or more processors; and
one or more computer-readable memories coupled to the one or more processors and having instructions stored thereon that are executable by the one or more processors to perform a method comprising:
receiving a target antibody sequence of a target antibody that includes a sequence of amino acids;
inputting the target antibody sequence into an antibody language model (ALM), wherein the ALM is a protein language model trained from antibody sequences, and the ALM comprises a plurality of self-attention layers;
obtaining, using the ALM without performing multiple sequence alignment (MSA), a residue encoding and an attention weight encoding, wherein:
the residue encoding comprises a respective first embedding corresponding to each of the amino acids in the target antibody sequence output by the ALM; and
the attention weight encoding comprises a respective second embedding corresponding to a pair of amino acids in the target antibody sequence calculated from attention weights of the self-attention layers of the ALM;
transforming the residue encoding and the attention weight encoding into a single representation and a pair representation;
inputting the single representation and the pair representation into a structure prediction model, wherein parameters of the structure prediction model are trained based on a loss function reflecting a difference between a predicted structure and an actual structure of an antibody;
determining the predicted structure of the target antibody using the structure prediction model based on the single representation and the pair representation; and
outputting the predicted structure of the target antibody.

12. The system of claim 11, wherein the ALM is pre-trained using an antibody database according to a Bidirectional Encoder Representations from Transformers (BERT) architecture, and the antibody database consists the antibody sequences.

13. The system of claim 11, wherein the ALM comprises L self-attention layers, each of the L self-attention layers comprises H attention heads, and
wherein a second embedding qᵢⱼ corresponding to a pair of an amino acid i and an amino acid j in the target antibody sequence, and
wherein obtaining, using the ALM without performing MSA, the attention weight encoding comprises:
obtaining attention weights of the H attention heads of the each of the L self-attention layers when the amino acid i is used as a query and the amino acid j is used as a key; and
concatenating the attention weights to obtain the second embedding qᵢⱼ.

14. The system of claim 11, wherein transforming the residue encoding and the attention weight encoding into a single representation and a pair representation comprises:
transforming the residue encoding into the single representation by a first linear neural network layer; and
transforming the attention weight encoding into the pair representation by a second linear neural network layer;
wherein parameters of the first linear neural network layer and the second linear neural network layer are updated based on a gradient of the loss function.

15. The system of claim 11, wherein, before inputting the single representation and the pair representation into the structure prediction model, the method further comprises:
performing a template search, based on the target antibody sequence without multiple sequence alignment (MSA), for one or more template candidates that have similar structures to a structure of the target antibody; and
obtaining template features based on the one or more template candidates; and
wherein transforming the residue encoding and the attention weight encoding into the single representation and the pair representation comprises:
transforming the residue encoding and the attention weight encoding into a preliminary single representation and an preliminary pair representation; and
incorporating the template features into the preliminary single representation and the preliminary pair representation to obtain the single representation and the pair representation.

16. One or more non-transitory, computer-readable media storing one or more instructions executable by a computer system to perform operations comprising:
receiving a target antibody sequence of a target antibody that includes a sequence of amino acids;
inputting the target antibody sequence into an antibody language model (ALM), wherein the ALM is a protein language model trained from antibody sequences, and the ALM comprises a plurality of self-attention layers;
obtaining, using the ALM without performing multiple sequence alignment (MSA), a residue encoding and an attention weight encoding, wherein:
the residue encoding comprises a respective first embedding corresponding to each of the amino acids in the target antibody sequence output by the ALM; and
the attention weight encoding comprises a respective second embedding corresponding to a pair of amino acids in the target antibody sequence calculated from attention weights of the self-attention layers of the ALM;
transforming the residue encoding and the attention weight encoding into a single representation and a pair representation;
inputting the single representation and the pair representation into a structure prediction model, wherein parameters of the structure prediction model are trained based on a loss function reflecting a difference between a predicted structure and an actual structure of an antibody;
determining the predicted structure of the target antibody using the structure prediction model based on the single representation and the pair representation; and
outputting the predicted structure of the target antibody.

17. The one or more non-transitory, computer-readable media of claim 16, wherein the ALM is pre-trained using an antibody database according to a Bidirectional Encoder Representations from Transformers (BERT) architecture, and the antibody database consists the antibody sequences.

18. The one or more non-transitory, computer-readable media of claim 16, wherein the ALM comprises L self-attention layers, each of the L self-attention layers comprises H attention heads, and
wherein a second embedding qᵢⱼ corresponding to a pair of an amino acid i and an amino acid j in the target antibody sequence, and
wherein obtaining, using the ALM without performing MSA, the attention weight encoding comprises:
obtaining attention weights of the H attention heads of the each of the L self-attention layers when the amino acid i is used as a query and the amino acid j is used as a key; and
concatenating the attention weights to obtain the second embedding qᵢⱼ.

19. The one or more non-transitory, computer-readable media of claim 16, wherein transforming the residue encoding and the attention weight encoding into a single representation and a pair representation comprises:
transforming the residue encoding into the single representation by a first linear neural network layer; and
transforming the attention weight encoding into the pair representation by a second linear neural network layer;
wherein parameters of the first linear neural network layer and the second linear neural network layer are updated based on a gradient of the loss function.

20. The one or more non-transitory, computer-readable media of claim 16, wherein, before inputting the single representation and the pair representation into the structure prediction model, the operations further comprise:
performing a template search, based on the target antibody sequence without multiple sequence alignment (MSA), for one or more template candidates that have similar structures to a structure of the target antibody; and
obtaining template features based on the one or more template candidates; and
wherein transforming the residue encoding and the attention weight encoding into the single representation and the pair representation comprises:
transforming the residue encoding and the attention weight encoding into a preliminary single representation and an preliminary pair representation; and
incorporating the template features into the preliminary single representation and the preliminary pair representation to obtain the single representation and the pair representation.
